(19) United States
(12) Patent Application Publication
(10) Pub. No.: US 2009/0092664 A1
(43) Pub. Date: Apr. 9, 2009

(54) POLYMER-METAL CHELATOR CONJUGATES AND USES THEREOF

(75) Inventors: Russell J. Mumper, Chapel Hill, NC (US); Anshul Gupte, Greenville, NC (US); Saurabh Wadhwa, Chapel Hill, NC (US)

Correspondence Address: FULBRIGHT & JAWORSKI L.L.P., 600 CONGRESS AVE., SUITE 2400, AUSTIN, TX, 78701, US

(73) Assignee: UNIVERSITY OF KENTUCKY RESEARCH FOUNDATION

(21) Appl. No.: 12/243,671

(22) Filed: Oct. 1, 2008

Related U.S. Application Data
(60) Provisional application No. 60/978,356, filed on Oct. 8, 2007.

(51) Int. Cl. A61K 9/127; C07K 19/00; C08B 37/08; C07K 2/00; A61K 38/17

(52) U.S. Cl. 424/450; 530/350; 536/20; 530/300; 530/391.7; 514/12

(57) ABSTRACT

The present invention provides prodrugs comprising a polymer conjugated to a metal chelator via a disulfide bond. For example, D-penicillamine may be conjugated to a polymer (e.g., gelatin, chitosan, polyglutamic acid) via a linker, such as SPDP. Thus, the cellular delivery and pharmacokinetics of D-penicillamine can be substantially improved. Methods for the treatment of cancer using compositions of the present invention are also disclosed.

This application claims priority to U.S. Application No. 60/978,356 filed on Oct. 8, 2007, the entire disclosure of which is specifically incorporated herein by reference in its entirety without disclaimer.

### BACKGROUND OF THE INVENTION

1. Field of the Invention

The present invention relates generally to the fields of molecular biology and medicine. More particularly, it concerns compositions and methods for the treatment of cancer.

2. Description of Related Art

Therapeutic selectivity (i.e., the degree to which a therapeutic can selectively target cancer cells as compared to normal cells) and drug resistance are two important factors that significantly affect the chances of a successful cancer therapy. In an effort to improve therapeutic selectivity and/or overcome drug resistance for cancer therapies, scientists have attempted to identify critical biochemical differences between cancer cells and normal cells and develop strategies that utilize these differences (Pelicano et al., 2004).

Oxidative stress is one biochemical difference between cancerous and non-cancerous cells which may be utilized by anti-neoplastic therapeutics. Strong evidence suggests that cancer cells, unlike healthy cells, are under an increased level of reactive oxygen species (ROS) stress (Behrend et al., 2003; Hileman et al., 2003; Zhou et al., 2003; Pelicano et al., 2003). Oxidative stress occurs when the production of ROS exceeds their removal with anti-oxidant compounds or enzymes (Leonard et al., 2005). Cellular defenses against ROS include anti-oxidants scavengers such as glutathione, ascorbate, thioredoxin and enzymes including catalase, superoxide dismutase, and glutathione peroxidase (Pelicano et al., 2004). Additionally, it has been suggested that the ability of a cell to defend itself against ROS is associated with resistance against chemotherapy (Renschler, 2004; Pervaiz and Clement, 2004). Several chemically diverse compounds can generate ROS and exhibit anti-cancer activity alone or in combination with other chemotherapeutic agents (Chen et al., 2005; Maeda et al., 2004; Okroj et al., 2006).

D-penicillamine (D-pen) is a copper chelating agent that is currently approved by the FDA for the treatment of Wilson's disease and rheumatoid arthritis. In recent years there has been substantial interest in use of copper chelators including D-pen as anti-angiogenic agents (Brem et al., 2005; Brewer, 2005).

Unfortunately, however, D-pen suffers from a number of unfavorable physicochemical properties which limit its potential as successful anti-cancer agent. These properties include: 1) D-pen is extremely hydrophilic (Log P: −0.39) (Chvapil et al., 2005) thus limiting its intracellular uptake in cancer cells (Joyce, 1989; Joyce, 1990); 2) D-pen is rapidly eliminated from the blood exhibiting biphasic kinetics (e.g., half-lives of 6-7 and 52-55 min) (Lu and Combs, 1992; Netter et al., 1987); and 3) the thiol group of D-pen which is critical to both copper chelation and H₂O₂ generation is prone to oxidation resulting in inactive D-pen disulfide or mixed disulfides both in-vitro and in-vivo. Therefore, the success of D-pen as anti-cancer agent would depend on its delivery to cancer cells in its reduced form or a modified form which could then be converted intracellularly to bioactive D-pen.

Attempts to improve D-pen have been made, with limited success. Chvapil et al. recently reported the synthesis of a hexyl-D-pen-ester (Log P: 1.61), which converted D-pen to a lipophilic form, but slowed the release of D-pen. Unfortunately, this ester prodrug strategy suffers from the lack of protection of the abovementioned critical thiol group of D-pen and also further suffers from the possibility that D-pen may be released from the ester at concentrations below minimum effective levels in-vivo.

Other compositions and compounds have been described which contain penicillamine. U.S. Application 2005/0080132 by Chvapil et al. describes admixtures of a lipophilic lathyrogen, such as penicillamine, and a polymeric carrier wherein the lipophilic lathyrogen in dispersed within the polymeric carrier. U.S. Application 2007/0072800 by Gengrinovitch et al. describes amino acids which may be attached to pharmacologically active compounds including penicillamine. U.S. Applications 2003/0147844 and 2003/0157052 by Choe et al. describe polymers attached to sulfhydryl containing moieties, such as penicillamine. Clearly, there exists a need for new compositions and methods for delivery of anti-neoplastic metal chelators.

### SUMMARY OF THE INVENTION

The present invention overcomes limitations in the prior art by providing novel compositions and methods for delivering an anti-neoplastic metal chelator, such as D-pen, to a cancerous cell. In various embodiments, compositions are provided which comprise a metal chelator, such as D-pen, conjugated to a polymer (e.g., gelatin, chitosan, polyglutamic acid). The metal chelator may be directly covalently bound to the polymer or indirectly covalently bound to the polymer via a linker, such as SPDP.

The metal chelator is preferably conjugated to the polymer via a disulfide bond which may be cleaved intracellularly and, in certain embodiments, comprises the sulfur group from the thiol of D-pen. Using a disulfide bond to conjugate a metal chelator such as D-pen and a polymer can allow for certain pharmacological advantages including: 1) protection of the thiol group of D-pen from oxidation before it reaches the site of action; 2) intracellular reversibility of binding (e.g., due to the presence of ˜1-11 mM of glutathione); and 3) the relative stability of disulfide bonds in plasma. Thus, the pharmacokinetics, potency, efficacy and/or cellular delivery of D-pen can be substantially improved according to the present invention. The present invention also provides methods for treating a tumor or cancer comprising administering a compound of the present invention to a subject, such as a human patient.

The present invention provides, in certain embodiments, a polymer conjugated to one or more D-pen. For example, D-pen may be covalently coupled to gelatin with a reversible disulfide with the aid of a heterobifunctional crosslinker, (N-Succinimidyl-3-(2-pyridyldithio)-propionate) (SPDP). As shown in the below examples, cell cytotoxicity and intracellular uptake was demonstrated in cancerous cells exposed to copper, using a human leukemia cell line (HL-60). In various embodiments, the degree of SPDP modification of gelatin amino groups may be about 50% when the amount of SPDP/gelatin is increased to about 0.23 (% w/w).

An aspect of the present invention relates to a composition comprising at least one D-penicillamine covalently bonded to a biocompatible polymer via a disulfide bond, wherein the disulfide bond comprises the sulfur group of D-penicillamine. The polymer may be gelatin, chitosan, or polyglutamic acid. In certain embodiments, the polymer is selected from the group consisting of poly-L-lysine, poly-L-Arginine, albumin, N-(2-hydroxypropyl) methacrylamide (HPMA), polyaspartamide, a dendrimer comprising a polyamido amine and polylysine core, hyaluronic acid, polylactic-co-glycolic acid, heparin, polyacrylic acid, crosslinked polyacrylic acid, carboxymethylcellulose, alginate, alginic acid, propylene glycol alginate, sodium alginate, a polylactide, poly-glutamic acid, and polyerucic-co-sebacic acid.

In certain embodiments said disulfide bond comprises a sulfur group present in the polymer, and wherein the D-penicillamine is covalently bonded directly to the polymer via the disulfide bond. In other embodiments, said disulfide bond comprises a sulfur group in a linker or a coupling agent, wherein the linker or coupling agent is covalently bonded to the polymer. Said linker may be selected from the group consisting of SPDP, LC-SPDP, and Sulfo-LC-SPDP. The polymer may have at least about 20% or has at least about 50% of available functionalities occupied by D-penicillamine or a linker, wherein the linker is coupled to D-penicillamine. The composition may comprise gelatin-D-penicillamine, chitosan-D-penicillamine, or polyglutamic acid-D-penicillamine.

In various embodiments, the polymer is conjugated to an antibody. The antibody may selectively bind a protein whose expression is upregulated in a cancer. The antibody may be CD123, Rituximab, Trastuzumab, Gemtuzamab, Alemtuzumab, Ibritumomab, Tositumomab, or Bevacizumab. The polymer may be conjugated to a polyethylene glycol having a molecular weight between about 2000 g/mol and about 20,000 g/mol.

In certain embodiments, the polymer is conjugated to an imaging agent. The imaging agent may be a fluorophore or a radioisotope. For example, the imaging agent may be a photon emission computed tomography (PET) imaging agent or a single photon emission computed tomography (SPECT) imaging agent.

The composition is preferably comprised in a pharmaceutically acceptable excipient. The pharmaceutically acceptable excipient may comprise a lipid, liposomes, or nanoparticles. The pharmaceutically acceptable excipient may be formulated for parenteral administration, intravenous administration, or intratumoral injection.

Another aspect of the present invention relates to a method of treating a cancer comprising administering a composition of the present invention to a subject, such as a mammal or a human patient. The composition may be administered parenterally, intravenously, or intratumorally. In certain embodiments, the composition is administered at a dose of from about 1 microgram/kg body weight to about 1000 milligram/kg body weight.

The method may further comprise administering a second cancer therapy to the subject. The second cancer therapy may be a chemotherapeutic, a surgery, a radiation therapy, an immunotherapy, or a gene therapy. The second cancer therapy may be paclitaxel, docetaxel, doxorubicin, a platinum-containing chemotherapeutic, idarubicin, or 5-FU. The cancer may be leukemia, cancer of the lymph node or lymph system, bone cancer, cancer of the mouth or esophagus, stomach cancer, colon cancer, breast cancer, ovarian cancer, a gastric cancer, brain cancer, renal cancer, liver cancer, prostate cancer, melanoma, or lung cancer.

“Conjugation,” as used herein, refers to the chemical attachment of two compounds, preferably via a covalent bond. The compounds may be directly conjugated or indirectly conjugated, such as via a linker compound. For example, a polymer may be covalently attached to a metal chelator via a disulfide bond; the metal chelator may be directly covalently attached to the polymer, or the metal chelator may be indirectly attached, e.g., covalently attached to a linker or coupling agent which is covalently attached to the polymer.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B: The concentration dependent in-vitro cytotoxicity of H₂O₂ alone compared to D-pen plus cupric sulfate in HL-60, HL-60/VCR and HL-60/ADR leukemia cells. FIG. 1A, H₂O₂ alone. FIG. 1B, D-pen plus cupric sulfate. Cells (3×10⁴ cells/well) were treated with increasing concentrations of H₂O₂ alone and D-pen plus cupric sulfate (10 μM) for 48 h in media. Cell viability was determined with the MTT assay. Absorbance was measured at 595 nm and adjusted to the absorbance of untreated cells. Data represent mean±SE; n=12 (two independent experiments performed in n=6). Inhibitory concentrations (IC₅₀) were reported as the mean±S.E in the figure legend.

FIGS. 2A-B: The concentration dependent in-vitro cytotoxicity of H₂O₂ alone compared to D-pen plus cupric sulfate in MCF-7 and BT474 breast cancer cells. FIG. 2A, H₂O₂ alone. FIG. 2B, D-pen plus cupric sulfate. Cells (3×10⁴ cells/well) were treated with increasing concentration of H₂O₂ and D-pen plus cupric sulfate for 48 h in media. Cell viability was determined with MTT assay. Absorbance was measured at 595 nm and adjusted to the absorbance of untreated cells. Data represent mean±SE; n=12 (two independent experiments performed in n=6). Inhibitory concentration (IC₅₀) was reported as mean±S.E.

FIGS. 3A-B: Catalase inhibits the D-pen plus cupric sulfate cytotoxicity in breast cancer and leukemia cells. FIG. 3A, MCF-7 cells. FIG. 3B, HL-60 cells. Cells (3×10⁴/well) were treated with D-pen, D-pen plus cupric sulfate (10 μM) and D-pen plus cupric sulfate (10 μM) plus catalase (500 U/mL) for 48 h in media. Cell viability was determined with MTT assay. Absorbance was measured at 595 nm and adjusted to the absorbance of untreated cells. Data represent the mean±SE; n=12 (two independent experiments performed in n=6). *P<0.05 and ***P<0.001 for D-pen plus cupric sulfate compared to D-pen alone and D-pen plus cupric sulfate plus catalase.

FIGS. 4A-B: D-pen in the presence of cupric sulfate generates intracellular ROS in breast cancer and leukemia cells. FIG. 4A, MCF-7 cells (3×10⁴/well) were loaded with ROS probe H₂DCFDA (5 μM). *P<0.05 for D-pen plus cupric sulfate (10 μM) compared to D-pen alone. ***P<0.001 for H₂O₂ compared to D-pen alone. FIG. 4B, HL-60 cells (3×10⁴/well) were loaded with ROS probe H₂DCFDA (5 μM). **P<0.01 D-pen plus cupric sulfate compared to D-pen alone. ***P<0.001 for H₂O₂ compared to D-pen alone. Cells were incubated without (positive control) or with H₂O₂, H₂O₂ plus catalase, cupric sulfate, D-pen, D-pen plus cupric sulfate, D-pen plus cupric sulfate plus catalase in media. Unloaded cells were used as negative control. The cell associated fluorescence was measured at 30, 60, 90 min at excitation and emission of 485±20 nm and 530±25 nm, respectively. Data are reported as the DCF fluorescence (fold increase versus control). Data represent mean±SE; n=12 (two independent experiments performed in n=6).

FIGS. 5A-B: The correlation between D-pen concentration and the intracellular ROS generation in breast cancer and leukemia cells. FIG. 5A, MCF-7 cells (3×10⁴/well) were loaded with ROS probe H₂DCFDA (5 μM). FIG. 5B, HL-60 cells (3×10⁴/well) were loaded with ROS probe H₂DCFDA (5 μM). Cells were incubated without (positive control) or with 50, 100 and 200 μM D-pen plus cupric sulfate (10 μM) in media. Unloaded cells were used as negative control. The cell associated fluorescence was measured at 30, 60, 90 and 120 min at excitation and emission of 485±20 nm and 530±25 nm, respectively. Data are reported as Fluorescence Units or the raw fluorescence of DCF. Data represent the mean±SE; n=12 (two independent experiments performed in n=6).

FIG. 6: D-pen in the presence of cupric sulfate causes the reduction in intracellular thiol levels in leukemia cells. HL-60 cells (5×10⁶/well) were plated in a 12 well plate and treated with D-pen plus cupric sulfate (10 μM) for 4 and 48 h. Cells were counted with trypan blue assay, washed with PBS buffer and homogenized in lysis buffer. Cells were centrifuged and the supernatant was used for total intracellular protein and thiol assay. Data represent the mean±SE; n=6 (two independent experiments performed in triplicate). *P<0.05 and **P<0.01 for D-pen (200 μM) plus cupric sulfate (10 μM) compared to control.

FIG. 7: Effect of increasing SPDP concentration on the modification of gelatin amine groups and moles of SPDP conjugated to gelatin. Pyridine-2-thione assay was used to determine the SPDP modification of gelatin. TNBS assay were used to determine the level of SPDP modification of gelatin. Data represents mean±S.E (n=3).

FIGS. 8A-B: Release of D-pen from the gelatin-D-pen conjugate in reducing conditions. FIG. 8A, The gelatin-D-pen conjugate was incubated with glutathione (1 mM) at 37° C. in phosphate buffer saline (PBS, pH 7.4 and pH 6.2) and the release of D-pen was analyzed with HPLC. Data represents mean±S.E (n=3). FIG. 8B, The gelatin-D-pen conjugate was incubated with increasing concentrations of glutathione (0, 0.1, 1, 10 mM) at 37° C. for 2 h. D-pen release was analyzed with HPLC. Data represents mean±S.E (n=3).

FIG. 9: D-pen release from conjugate in presence of DTT. The conjugate was incubated with increasing concentration of DTT in PBS, pH 7.4 at 37° C. for 2 h. D-pen release was quantified with the HPLC assay.

FIG. 10: In vitro Cytotoxicity of gelatin alone in human leukemia cells (HL-60). HL-60 cells (20,000/well) were treated with media alone (control) and Gelatin (μg/mL). MTT assay was conducted at 24 and 72 h. Data is reported n=3±S.E.

FIGS. 11A-B: In vitro Cytotoxicity of Gelatin and D-pen in presence of cupric sulfate (10 μM) human leukemia cells (HL-60). HL-60 cells (20,000/well) were treated with media+CuSO4 (10 μM) (control) and Gelatin (μg/mL)+CuSO4 (10 μM) or D-pen (μM)+CuSO4 (10 μM). MTT assay was conducted at 24 and 72 h. Data is reported n=3±S.E.

FIG. 12: Fluorescent labeling of Gelatin-D-pen conjugate for cell uptake and association studies.

FIG. 13: Cell viability of cupric sulfate (0 μM) pre-treated cells. ***P<0.001 compared to 2000 μg/mL gelatin alone

FIG. 14: Cell viability of cupric sulfate (10 μM) pre-treated cells. ***P<0.001 compared to gelatin alone at corresponding concentration

FIG. 15: Cell viability of cupric sulfate (25 μM) pre-treated cells. *P<0.05, **P<0.01, ***P<0.001 compared to gelatin alone at corresponding concentration.

FIG. 16: Cell viability of cupric sulfate (50 μM) pre-treated cells. **P<0.01, ***P<0.001 compared to gelatin alone at corresponding concentration.

FIG. 17: Cell viability of cupric sulfate (100 μM) pre-treated cells.

FIG. 18: Enhancement of D-pen cytotoxicity by copper pre-treatment of human leukemia cells (HL-60). **P<0.01, ***P<0.001 for D-pen alone vs. cupric sulfate pre-treated HL-60 cells and D-pen+cupric sulfate vs. HL-60 cells compared to D-pen alone vs. HL-60 cells. Cell viability was assessed with MTT assay after 48 h incubation.

FIG. 19: Cellular uptake of D-pen. D-pen (100 μM) was added to HL-60 (1×10⁶) cells in PBS (pH 7.4) in a 24-well plate at 37° C. in 5% CO₂ incubator. Cells were separated by centrifugation and the supernatant was analyzed by HPLC assay for D-pen concentration. Data represents mean±SD (n=4).

FIG. 20: Cytotoxicity of the gelatin-D-pen conjugate in HL-60 cells. Cells (2×10⁴) were incubated with gelatin alone (1 mg/mL), D-pen alone (100 μM), gelatin (1 mg/mL) plus D-pen (100 μM), and gelatin-D-pen conjugate (1 mg/mL) After every 2 day interval media was replaced with fresh media. MTT assay was performed on day 2, 4, 6, 8 and 10. Data represents mean±SD (n=6). **p<0.01 and ***p<0.001 compared to gelatin plus D-pen.

FIG. 21: In vitro cytotoxicity of PGA-Dpen conjugate at 48 hr in HL-60 cells (triangles), P388 cells (squares), and MDA-MB-468 cells (diamonds). The log of equivalent D-pen concentration was plotted on the X-axis.

FIG. 22: Intracellular ROS generation by PGA-D-pen conjugate in HL-60 cells. Cells were incubated with 25 μM carboxy-H2DCFDA for 30 min before exposure to PGA-D-pen conjugate. 100 μM H2O2 was used as positive control. The fluorescence values were measured at 8 h after treatment.

### DESCRIPTION OF ILLUSTRATIVE EMBODIMENTS

The present invention provides compositions and methods for delivering an anti-neoplastic metal chelator, such as D-pen, to a cancerous cell. For example, D-pen may be conjugated to a polymer (e.g., gelatin, chitosan, polyglutamic acid) via a disulfide bond. The disulfide bond may be cleaved intracellularly, releasing D-pen into the cell. In the presence of copper, D-pen can generate cytotoxic reactive oxygen species and cause cell death. Since various cancers contain increased levels of copper relative to non-cancerous cells, the cancerous cells (e.g., in a solid tumor, or a leukemia, etc.) may be selectively killed. Thus, the cellular delivery and pharmacokinetics of D-pen can be substantially improved according to the present invention. For example, as shown in the below examples, D-pen polymer conjugates can selectively kill cancerous cells in the absence of copper and display superior therapeutic activities as compared to the simultaneous administration of both D-pen and a polymer. The polymer-metal chelator conjugates of the present invention may be administered to a subject such as a human patient to treat a cancer.

### I. Anti-Angiogenic Metal Chelators

Certain metal chelators which may be used with the present invention generate reactive oxygen species (ROS) intracellularly. Cancer cells differ from typical cells as they exhibit increased intrinsic ROS stress due to a number of factors including the oncogenic stimulation, increased metabolic activity, and mitochondrial malfunction (Pelicano et al., 2004; Renschler, 2004; Leonard et al., 2005). As a result, cancer cells under sustained ROS stress conditions tend to heavily utilize adaptation mechanisms and may exhaust ROS-buffering capacity while normal cells have low levels of ROS stress and reserve a higher capacity to cope with further oxidative insults (Renschler, 2004; Leonard et al., 2005). Therefore, the generation of ROS can be exploited therapeutically in the treatment of cancer (Renschler, 2004). Several anticancer agents currently employed in cancer treatment including anthracyclines, bleomycin, and cisplatin are known to either generate cellular ROS or to impair the cellular redox buffering (Pelicano et al., 2004; Schafer and Buettner, 2001).

Copper metal chelators, such as D-penicillamine, may be particularly useful with the present invention. D-pen is a potent copper chelating agent that has been investigated in the recent years as a potential anti-angiogenic agent based on its efficient copper chelating and removing abilities (Brem et al., 2005; Brem et al., 1999).

Copper plays a critical role in cancer and is important for certain anti-angiogenic agents which utilize increased copper concentrations in neoplastic cells. Copper has been established as a key co-factor required by a number of pro-angiogenic molecules including fibroblast growth factor (FGF) (Mamou et al., 2006), vascular endothelial growth factor (VEGF) (Pan et al., 2002), and interleukin-1 (Pan et al., 2002). Several in-vitro studies have shown that high copper concentrations facilitate the proliferation of cancer cells (Lowndes and Harris, 2004; Guo, 1998, Raju et al., 1982). Serum and tumor copper levels have been shown to be significantly elevated in breast cancer (Santoliquido et al., 1976; Schwartz et al., 1974; Yucel et al., 1994), lung cancer (Diez et al., 1989; Scanni et al., 1977), leukemia (Zuo et al., 2006), and gynecological cancer (Chan and Wong, 1993). Thus, anti-copper therapy has been investigated as an anti-angiogenic strategy for cancer treatment (Brem et al., 2005; Brewer, 2005) and include copper chelating agents, tetrathiomolybdate (Mamou et al., 2006; Pan et al., 2002), clioquinol (Daniel et al., 2005; Ding et al., 2005), and D-pen (Brem et al., 2005; Brewer, 2005; Lowndes and Harris, 2004).

Certain metal chelators of the present invention generate reactive oxygen species (ROS) in response to metal chelation. Thiol containing compounds are known to be cytotoxic at moderate concentrations but not at low and high concentrations (Held et al., 1996; Held and Biaglow, 1994). At low thiol concentration, very small levels of ROS are produced and the cells possess sufficient anti-oxidant capacity to defend themselves against this ROS stress (Munday, 1989). At high concentrations, thiols react with the generated H₂O₂ and other ROS and thus act as anti-oxidants (Munday, 1989). It has been shown that the rate of thiol reaction with H₂O₂ and superoxide is inversely related to its pKa (Winterbourn and Metodiewa, 1999). Therefore, thiol toxicity depends on the interplay between the rate of transition metal (copper, iron) catalyzed thiol oxidation and the rate of thiol reaction with H₂O₂ and other ROS generated during thiol oxidation (Held and Biaglow, 1994). Thiolate ion plays a critical role in metal catalyzed thiol oxidation (Starkebaum and Root, 1985).

Other metal chelators may be used with the present invention including, but not limited to, desferrioxamine, Triapine™ (3-aminopyridine-2-carboxaldehyde thiosemicarbazone), trientine, clioquinol, tetrathiomolybdate, and/or thioctic (alpha-lipoic) acid. Generally, chelators that bind endogenous metals such as copper, zinc, or iron to produce H₂O₂ and/or other reactive oxygen species may be used with the present invention. It is envisioned that virtually any chelator with anti-angiogenic properties may be conjugated to a polymer according to the present invention.

A. D-Penicillamine

D-penicillamine (D-pen) is an aminothiol and a potent copper chelating agent (Vande Stat et al., 1979; Schilsky, 1996). D-pen is currently approved for the treatment of Wilson's disease and rheumatoid arthritis. Based on its ability to effectively chelate and remove copper, it has also been investigated as an anti-angiogenic agent (Vande Stat et al., 1979; Brem et al., 2005; Brewer, 2005; Lowndes and Harris, 2004). D-pen has the following structure:

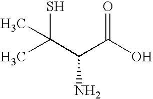

D-pen can be used according to the present invention as an anti-cancer agent. Serum and tumor copper levels are significantly elevated in a variety of malignancies including breast, ovarian, gastric, lung cancer and leukemia. D-pen at low concentrations in the presence of copper generates concentration dependent cytotoxic hydrogen peroxide (H₂O₂).

In the process of chelating copper, D-pen reduces Cu(II) to Cu(I) leading to the generation of hydrogen peroxide (H₂O₂) and other ROS (Matasubara et al., 1989; Samoszuk and Nguyen, 1996; Starkebaum and Root, 1985). D-pen has been shown to inhibit human endothelial cell proliferation in-vitro and neovascularization in-vivo (Matasubara et al., 1989), and suppress human fibroblast proliferation (Matasubara and Hirohata, 1988) in the presence of copper. Auto-oxidation of other thiols (cysteamine, homocysteine) in the presence of copper has also been shown to generate H₂O₂ and cause cytotoxicity (Nath and Salahudden, 1993; Nakanishi et al., 2005).

D-pen generates concentration dependent ROS only in the presence of copper and exhibits concentration dependent cytotoxicity in cancer cells. As demonstrated in the below examples, in-vitro cytotoxicity, intracellular ROS generation, and the reduction in intracellular thiol levels due to H₂O₂ and other ROS were generated from copper catalyzed D-pen oxidation in human breast cancer cells (BT474, MCF-7) and human leukemia cells (HL-60, HL-60/VCR, HL-60/ADR). D-pen (≦400 μM) in the presence of cupric sulfate (10 μM) resulted in concentration dependent cytotoxicity. Catalase was able to completely protect the cells, substantiating the involvement of H₂O₂ in cancer cell cytotoxicity. A linear correlation between the D-pen concentration and the intracellular ROS generated was shown in both breast cancer and leukemia cells. D-pen in the presence of copper also resulted in a reduction in intracellular reduced thiol levels. The H₂O₂-mediated cytotoxicity was greater in leukemia cells compared to breast cancer cells. Without wishing to be bound by any theory, D-pen exhibits cytotoxic effects based on its ROS generating ability in response to copper chelation.

### II. Polymers

The present invention provides effective D-pen delivery through the conjugation of D-pen to a macromolecular polymer. In various embodiments, the polymer preferably has the following qualities: i) a simple conjugation procedure can allow for conjugation of an anti-angiogenic metal chelator to the polymer; ii) the polymer is biocompatible; iii) the polymer is biodegradable; and iv) the conjugation of the metal chelator (e.g., D-pen) to the polymer is reversible.

Various polymers are known in the art, such as natural and synthetic polymers, which may be used with the present invention. The covalent conjugation of low molecular weight drug with soluble macromolecular carriers (peptides, protein and polymers) have been the focus of substantial research (Bowman and Ofner, 2000; Ofner et al., 2006).

A proportion of the available functionalities of the polymer can be occupied by a metal chelator such as D-pen or by a linker, wherein the linker is conjugated to a metal chelator such as D-pen. For example, a polymer may have from about 15% to about 75%, from about 15% to about 75%, or at least about 10%, 15%, 20%, 25%, 30%, 35%, 40%, 45%, 50%, 55%, 60%, 70%, 75%, 80%, 85%, 90%, 95%, or more, about 100%, or any range derivable therein of its available functionalities occupied by a metal chelator such as D-pen or by a linker, wherein the linker is conjugated to D-pen. In certain embodiments, D-pen is covalently bonded to the polymer or linker via a disulfide bond comprising the sulfur group of D-pen.

The following polymers listed below in Table 1 may be conjugated to a metal chelator such as D-penicillamine.

TABLE 1
Polymers for conjugation to D-penicillamine or other metal chelator:

Gelatin
Chitosan
Poly-L-lysine
Poly-L-ornithine, Poly-L-arginine and other polyamino acids
Polyaspartamide
Polyethylene glycol
Dextran
Hyaluronic acid
Dendrimer (polyamido amine or polylysine core)
Polyvinyl alcohol
Heparin
Polyacrylic acid, and crosslinked polyacrylic acid
Carboxymethylcellulose
Hydroxylethyl cellulose
Hydroxypropyl cellulose
Polyvinyl pyrrolidone
Polyhydroxybutyrate
Polystyrenesulfonate
Poly(methylidene malonate)
Polyotho esters
Poly(anhydride esters)
Polysulphone
Polyethylene imine
Polyerucic-co-sebacic acid
Poly(caprolactone)
Poly(ester-carbonate)
Cyclodextrin polymers
Poly(methyl vinyl ether-co-maleic anhydride)
Polyimidazole
Polystyrene
Polyphosphazene
Poly(ethylene oxide)
Poly(propylene oxide)
Copolymers of poly(ethylene oxide) and poly(propylene oxide)
Poly(hydroxybutyrate)
Poly(ester amide) copolymers
N-(2-hydroxypropyl) methacrylamide (HPMA)
Poly-glutamic acid
Albumin
Alginate, alginic acid, and propylene glycol alginate
Sodium Alginate
Polylactides
Polyglycolides
Polylactic-co-glycolic acid

In certain embodiments, gelatin is conjugated to a metal chelator according to the present invention. Gelatin is a partially hydrolyzed form of collagen (Bowman and Ofner, 2000). It has been used both in pharmaceutical and therapeutic applications (Young et al., 2005). Gelatin has been reported to be both biocompatible and biodegradable (Young et al., 2005; Kommareddy and Amiji, 2005). These and other distinctive physiochemical properties of gelatin make it a suitable candidate for the delivery of genes, chemotherapeutic agents as conjugates (Bowman and Ofner, 2000; Ofner et al., 2006; Chung et al., 2003; Pica et al., 2006), nanoparticles (Young et al., 2005; Azarmi et al., 2006; Kaul and Amiji, 2002; Kommareddy and Amiji, 2007) and microspheres (Young et al., 2005). Both the carboxyl and the amino groups of gelatin have been reported to be modified to conjugate drugs with gelatin (Kommareddy and Amiji, 2005; Pica et al., 2006).

It is envisioned that any antibody specific for a tumor cell may be further conjugated to a polymer of the present invention (e.g., a polymer conjugated to D-pen) to aid in targeting a cancer cell. Of particular interest are those antibodies or antibody fragments that preferentially target leukemia, cancer of the lymph node or lymph system, bone cancer, cancer of the mouth and/or esophagus, stomach cancer, colon cancer, breast cancer, ovarian cancer, a gastric cancer, brain cancer, renal cancer, liver cancer, prostate cancer, melanoma, and/or lung cancer. Specific antibodies of interest include, but are not limited to: CD123 (leukemia), Rituximab (Rituxan™), Trastuzumab (Herceptin™), Gemtuzamab (Mylotarg™), Alemtuzumab (Campath™), Ibritumomab (Zevalin™), Tositumomab (Bexxarm), and Bevacizumab (Avastin™).

It is also envisioned that other means of targeting to cancer cells including the use of small molecules such as, but not limited to, folate or transferrin, or other targeting moieties such as carbohydrates and peptides.

The polymer conjugate or polymer-antibody conjugate may be pegylated with polyethylene glycol to allow the conjugate to be retained for increased periods of time in circulation. In these embodiments, various size ranges of PEG may be used. For example, the molecular weight of the polyethylene glycol may be from about 1000 g/mol to 10,000 g/mol, more preferably from about 2000 g/mol to about 20,000 g/mol.

### III. Linkers/Coupling Agents

A metal chelator may be joined to a polymer via a linker or coupling agent which may be cleaved intracellularly or intratumorally. For example, a metal chelator such as D-penicillamine may be joined to a polymer (e.g., chitosan, gelatin, polyglutamic acid, etc.) via a disulfide bond; once the polymer-metal chelator is intracellular the disulfide bond may then be cleaved, releasing the metal chelator(s) from the polymer.

Conjugating a metal chelator such as D-pen to a polymer via a disulfide bond has advantages including: 1) protection of the thiol group of D-pen from oxidation before it reaches the site of action; 2) intracellular reversibility (e.g., due to the presence of ˜1-11 mM of glutathione) (Saito et al., 2003; Schafer and Buettner, 2001; Cavallaro et al., 2006); and 3) its relative stability in plasma (Jones et al., 2000).

A metal chelator may also be joined to a polymer via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metalloproteinase, such as collagenase, gelatinase, or stromelysin. Alternatively, peptides or polypeptides may be joined to an adjuvant.

Amino acids such as selectively-cleavable linkers, synthetic linkers, or other amino acid sequences may be used to separate a polymer and metal chelator. Additionally, while numerous types of disulfide-bond containing linkers are known that can successfully be employed to conjugate a polymer to a metal chelator, certain linkers will generally be preferred over other linkers, based on differing pharmacologic characteristics and capabilities. For example, linkers that contain a disulfide bond that is sterically “hindered” are to be preferred, due to their greater stability in vivo, thus preventing release of the metal chelator prior to entering the intracellular environment of a cell, such as a cancer cell.

Cross-linking reagents are used to form molecular bridges that tie together functional groups of two different molecules, e.g., a stabilizing and coagulating agent. To link two different proteins in a step-wise manner, hetero-bifunctional cross-linkers can be used that eliminate unwanted homopolymer formation.

It is contemplated that cross-linkers may be implemented with the polymer-metal chelators of the invention. Bifunctional cross-linking reagents have been extensively used for a variety of purposes including preparation of affinity matrices, modification and stabilization of diverse structures, identification of binding sites, and structural studies. In the context of the invention, such cross-linker may be used to stabilize the polymer-metal chelator conjugate or to render it more useful as a therapeutic, for example, by improving the polymer-metal chelator conjugate's targeting capability or overall efficacy. Cross-linkers may also be cleavable, such as disulfides, acid-sensitive linkers, and others. Homobifunctional reagents that carry two identical functional groups proved to be highly efficient in inducing cross-linking between identical and different macromolecules or subunits of a macromolecule, and linking of polypeptides to specific binding sites on binding partners. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites will be dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the chemistry of cross-linking, linkage of the amine residues of the recognizing substance and liposomes may be established. Compositions of the present invention may be present in a liposome or other particle such as a nanoparticle. A “nanoparticle” is defined as a particle having a diameter between about 1 and about 1000 nanometers; in certain embodiments, the nanoparticle may be from about 1 to about 500, from about 1 to about 100, or from about 10 to about 100 nanometers in size.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars. Table 2 details certain hetero-bifunctional cross-linkers considered useful in the present invention.

TABLE 2
HETERO-BIFUNCTIONAL CROSS-LINKERS

| | | | Spacer Arm |
|---|---|---|---|
| | | | Length\after |
| Linker | Reactive Toward | Advantages and Applications | cross-linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC-SPDP | Primary amines | Extended spacer arm | 15.6 A |
| | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo-SMCC | Primary amines | Stable maleimide reactive group | 11.6 A |
| | Sulfhydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo-SMPB | Primary amines | Extended spacer arm | 14.5 A |
| | Sulfhydryls | Water-soluble | |
| EDC/Sulfo-NHS | Primary amines | Hapten-Carrier conjugation | 0 |
| | Carboxyl groups | | |
| ABH | Carbohydrates | Reacts with sugar groups | 11.9 A |
| | Nonselective | | |

Additionally, any other linking/coupling agents and/or mechanisms known to those of skill in the art can be used to combine the components of the present invention, such as, for example, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

It is contemplated that a cross-linker having reasonable stability in blood will be employed. Numerous types of disulfide-bond containing linkers are known that can be successfully employed to conjugate targeting and therapeutic/preventative agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido)ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane. The use of such cross-linkers is well understood in the art. Another embodiment involves the use of flexible linkers.

Other linkers are known in the art and may be used in certain embodiments of the present invention. For example, U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions.

### IV. Synthesis

Several general synthesis methods may be utilized to conjugate a metal chelator to a polymer. These methods include amino modification, thiolation, and carboxyl modification. Various polymers may easily be conjugated to a metal chelator, such as D-pen, via these methods as shown below in Table 3.

TABLE 3

| Method I | Method II | Method III |
|---|---|---|
| Amino Modification | Thiolation | Carboxyl Modification |
| Gelatin | Gelatin | Hyaluronic acid |
| Chitosan | Chitosan | Polylactic-co-glycolic acid |
| Poly-L-lysine | Poly-L-lysine | Heparin |
| Poly-L-Arginine | Poly-L-Arginine | Polyacrylic acid, and |
| | | crosslinked polyacrylic acid |
| Albumin | Albumin | Carboxymethylcellulose |
| Dendrimer (polyamido | | Alginate, alginic acid, and |
| amine and polylysine | | propylene glycol alginate |
| core) | | |
| N-(2-hydroxypropyl) | N-(2- | Sodium Alginate |
| methacrylamide | hydroxypropyl) | |
| (HPMA) | methacrylamide | |
| | (HPMA) | |
| Polyaspartamide | Polyaspartamide | Polylactides |
| | | Poly-glutamic acid |
| | | Polyerucic-co-sebacic acid |

Syntheses which result in a disulfide bond being formed between a metal chelator, such as D-pen, and a polymer have certain advantages, as stated above. Specifically, the disulfide bond has substantial advantages for delivery of D-pen (e.g., from gelatin-D-pen) including: 1) protection of the thiol group of D-pen from oxidation before it reaches the site of action; 2) its intracellular reversibility (e.g., due to the presence of ˜1-11 mM of glutathione) (Saito et al., 2003; Schafer and Buettner, 2001; Cavallaro et al., 2006); and 3) its relative stability in plasma (Jones et al., 2000).

A. Amino Modification

This synthesis method involves thiolating the polymer and then conjugating a metal chelator (e.g., D-pen) via a disulfide bond. This strategy is illustrated below with a method for the conjugation of chitosan to D-pen; nonetheless, those of skill in the art will recognize that this synthesis is presented for illustrative purposes only and additional polymers, metal chelators, and optionally linkers may be used according to the below synthesis strategy.

Traut's reagent (2-iminothiolane) was used to attach thiol group to a biocompatible polymer, chitosan (amine containing biocompatible polymer). Traut's reagent is a cyclic thioimidate that reacts with primary amines (—NH₂) to introduce sulfhydryl (—SH) group, while maintaining the charge properties similar to the primary amino group.

The synthesis of chitosan-D-penicillamine conjugate may be performed in two steps. Briefly, traut's reagent is incubated with chitosan for 2 h in PBS, pH 7.4. Excess unreacted traut's reagent is separated by either centrifugation or dialysis. The thiolated chitosan is then incubated with D-penicillamine overnight to form a stable chitosan-D-pen disulfide. Excess D-pen is removed by centrifugation or dialysis.

B. Thiolation

This synthesis method involves reacting polymer —NH₂ with a bifunctional linker in order to conjugate a metal chelator, e.g., D-pen, to the polymer via a disulfide bond. This method is illustrated below with a method for the conjugation of gelatin to D-pen using SPDP; nonetheless, those of skill in the art will recognize that this synthesis is presented for illustrative purposes only and additional polymers, metal chelators, and/or linkers may be used according to the below synthesis strategy.

A heterobifunctional cross-linker (Sulfo-LC-SPDP) was employed to conjugate D-pen with gelatin (amine containing biocompatible polymer). Sulfo-SPDP has an amine reactive N-hydroxysuccinimide (NHS) and a thiol reactive 2-pyridylthio group. Generally, the criteria for selecting a heterobifunctional cross linkers used according to this method is that they have an amine reactive and a thiol reactive side. Additionally, the thiol reactive side should typically form a disulfide bond with D-penicillamine (SPDP, LC-SPDP, and Sulfo-LC-SPDP) and not the i) irreversible maleimide bond (SMCC, Sulfo-SMCC, MBS, Sulfo-MBS, SMPB and Sulfo-SMPB) and the ii) more stable thioether bond (SIAB, Sulfo-SIAB).

The synthesis of Gelatin-D-penicillamine conjugate may be performed in two steps. Briefly, gelatin is incubated with SPDP for 2 h at room temperature in PBS, pH 7.4. Excess SPDP is separated from gelatin with centrifugation or dialysis. The SPDP-modified gelatin is then incubated with D-pen overnight. The gelatin-D-pen conjugate is separated from the unconjugated D-pen with either centrifugation or dialysis.

One general synthesis strategy for the conjugation of a metal chelator to a polymer is shown below:

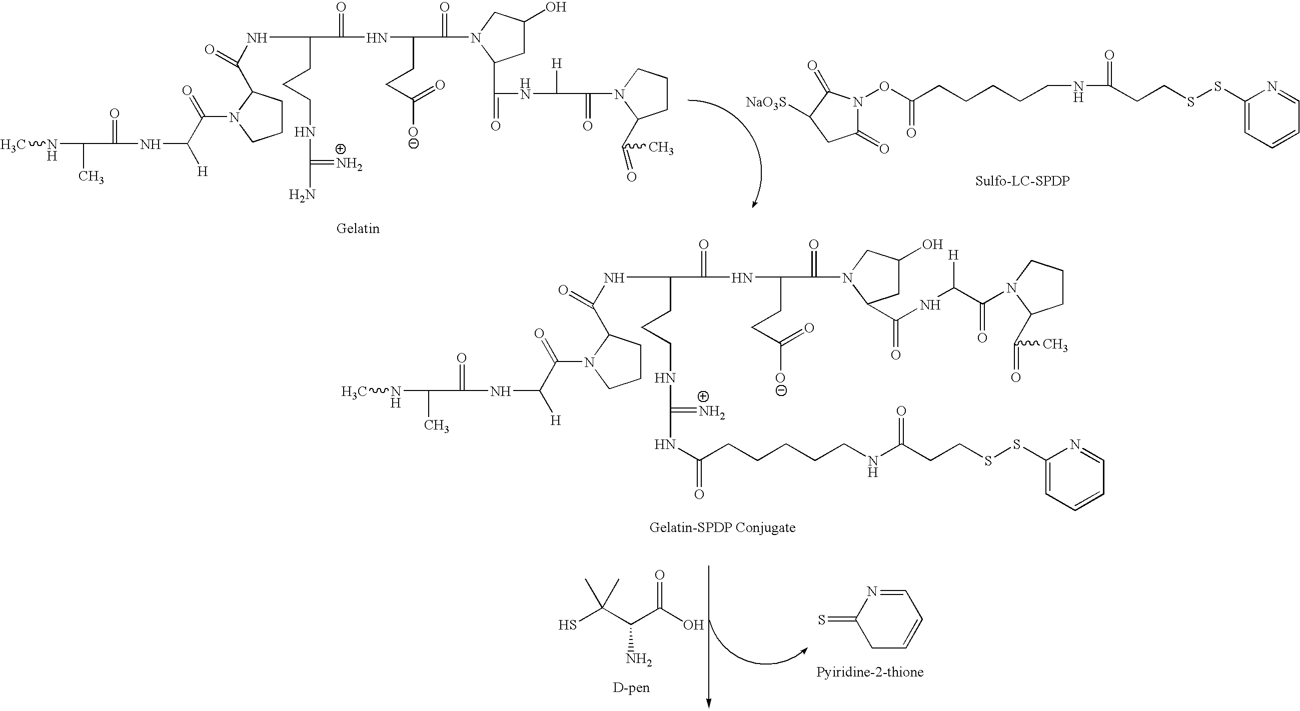

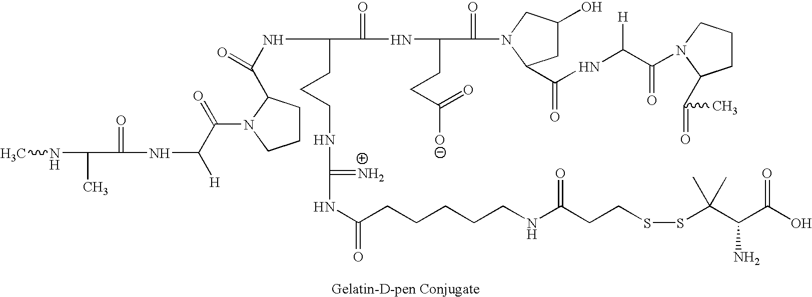

As shown above, the amino group of gelatin may be modified with the amine reactive N-hydroxysuccinimide (NHS) portion of Sulfo-SPDP and D-pen can then be conjugated to the SPDP-modified Gelatin by reacting with sulfhydryl reactive 2-pyridothione.

1. Synthesis of Gelatin D-pen

A soluble gelatin-D-pen conjugate may be synthesized, for example, through the modification of gelatin with the aid of a heterobifunctional cross-linker, sulfosuccinimidyl 6-[3′ (2-pyridyldithio)-propionamido]hexanoate Sulfo-LC-SPDP (water soluble derivative of SPDP). As described in the below examples, gelatin was coupled with SPDP through the amino group of gelatin and the amine-reactive N-hydroxysuccinimide (NHS) ester of SPDP to form a stable amide bond. D-pen was then conjugated to the SPDP modified gelatin through thiol exchange with D-pen; thus, D-pen was efficiently conjugated to gelatin with a reversible disulfide bond.

Specifically, D-pen may be conjugated to gelatin via the following method. A heterobifunctional cross-linker (Sulfo-SPDP) may be employed to conjugate D-pen with gelatin. Sulfo-SPDP has an amine reactive N-hydroxysuccinimide (NHS) and a thiol reactive 2-pyridylthio group. The synthesis may be performed in two steps. Firstly, gelatin (10 mg/mL) may be incubated with increasing amounts of SPDP for 2 h at room temperature in PBS, pH 7.4. Excess SPDP may be separated from gelatin with microcon centrifugation devices (MWCO: 10 kDa). Pyridine-2-thione and the TNBS assay may be performed to determine the degree of SPDP modification of gelatin. The SPDP-modified gelatin may then be incubated with D-pen. The gelatin-D-pen conjugate may be separated from the unconjugated D-pen using a microcon centrifugal device. A HPLC assay may be performed on the filtrate and retentate to determine the amount of D-pen conjugated to gelatin.

The complete and efficient separation of gelatin from unreacted SPDP or D-pen may be performed with microcon centrifugation tubes (MWCO: 10 kDa). For example, 500 μL of reaction mixture may be added to the microcon tubes, and the mixture may be centrifuged at 14,000 g for 30 min at room temperature. The filtrate may be collected and 400 μL of fresh PBS buffer can then added to the retentate. The centrifugation may be performed again at 14,000 g at 30 min. Filtrate may be collected and the retentate can then be collected by centrifuging at 3,000 g for 5 min. Both the filtrate and the retentate can then be analyzed. Gelatin may be detected and quantitated with coomassie, TNBS and the pyridine 2-thione assay. D-pen can then be detected via the HPLC assay.

The effect of increasing SPDP/gelatin (% w/w) on the D-pen (mmol/g gelatin and mg/g gelatin) conjugated to gelatin in the gelatin-D-pen conjugate is shown below in Table 3.

TABLE 3

| SPDP/gelatin | D-pen (mmol)/ | D-pen (mg)/ |
|---|---|---|
| (% w/w) | gelatin (g) ± S.D | gelatin (g) ± S.D |
| 0.02 | 20.5 ± 0.5 | 3.1 ± 0.07 |
| 0.06 | 52.8 ± 2.0 | 7.8 ± 0.3 |
| 0.1 | 58.8 ± 0.9 | 8.7 ± 0.14 |
| 0.18 | 84.3 ± 9.1 | 12.6 ± 1.3 |
| 0.23 | 102.4 ± 1.4 | 15.3 ± 0.2 |

C. Carboxyl Modification

A metal chelator may be conjugated to a polymer via a bifunctional linker by utilizing a reactive carboxy group on the bifunctional linker. This method may be achieved via the following steps: (1) Reacting carboxyl groups of a suitable polymer (e.g., polyglutamic acid) with the carboxy reactive group on a hetero-bifunctional linker to form an amide bond in presence or absence of a carboxy group activator like carbodiimide. (2) Isolation and purification of the hetero-bifunctional linker reacted polymer from the unreacted species. (3) Reacting amino group(s) of metal chelator with the sulfhydryl reactive group(s) of the purified hetero-bifunctional linker reacted polymer. And finally, (4) isolation and purification of the metal chelator reacted polymer, e.g., using a method as described above.

For example, the direct conjugation of polyglutamic acid and D-pen may be accomplished using the strategy illustrated below:

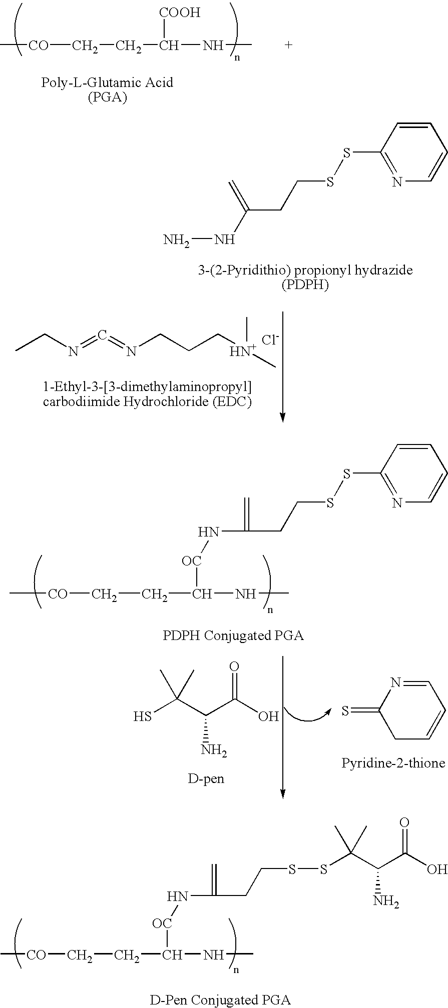

EDC is an activator of carboxylic acid group and the reaction should typically be done in the presence of a conjugating species like PDPH, as the intermediate O-acyl urea is short lived. EDC is also called a zero-length cross-linker. PDPH is a hetero-bifunctional cross-linker with a carboxy reactive end and a sulfhydryl reactive end. 0.1 M Morphoethanesulfonic acid buffer pH 4.5-5.0 can be used as a conjugation buffer. Using this approach, the PGA D-pen was determined to have 0.199 g D-pen or 1333 μM Dpen/g PGA.

In other embodiments, conjugation of polyglutamic acid and D-pen may be accomplished using the following strategy. PGA-D-pen conjugate may be synthesized as shown below. In the first step, cystamine may be covalently conjugated to PGA to form PGA-cystamide. D-pen may be conjugated to PGA-cystamide in the second step via thiol-disulfide exchange. N-hydroxy succinimide (NHS) (1.76 mg, 0.015 mmol), 1-Ethyl-3-[3-dimethylaminopropyl]carbodiimide Hydrochloride (EDC) (29.39 mg, 0.153 mmol), triethyl amine (1 mmol) and cystamine dihydrochloride (34.45 mg, 0.153 mmol) may then be added to a solution of sodium PGA (20 mg, 0.67 μmol) in DMF-H₂O (4/1). The reaction mixture may be stirred for 2 h at room temperature. The solvent may be removed by vacuum evaporation, the mixture reconstituted in 0.05 M Borate buffer pH 9.0, and PGA-cystamide may be purified using a Sephadex G-25 column. D-pen (34.32 mg, 0.23 mmol) may be added to PGA-cystamide solution in 0.05 M Borate buffer pH 9.0 and the reaction mixture may be left under constant stirring for 16 h at room temperature. PGA-D-pen conjugate may be purified using a Sephadex G-25 column.

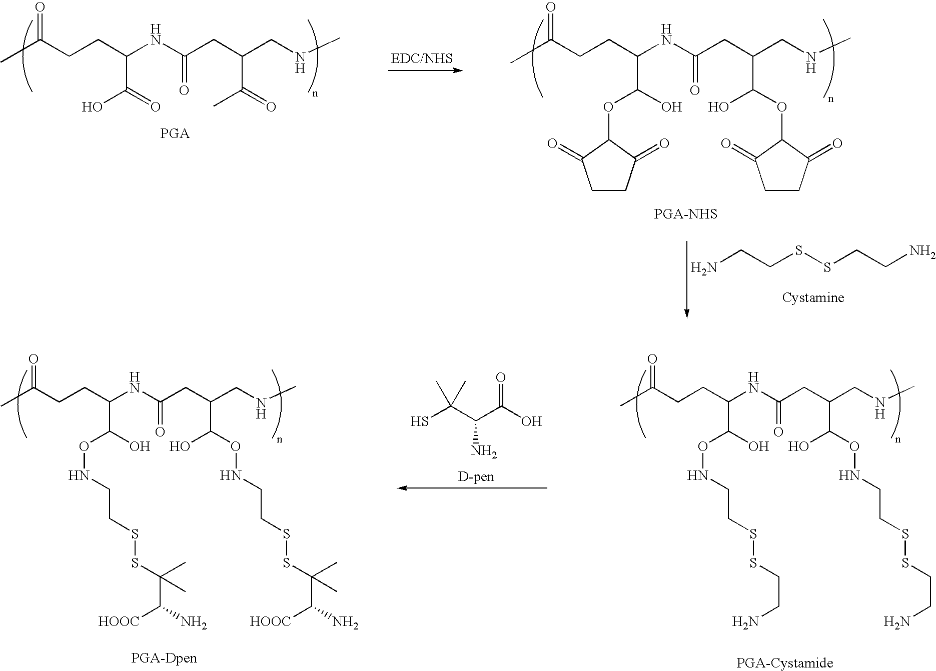

The above synthesis of PGA-D-pen conjugate utilizes the following abbreviations: PGA, Poly-1-glutamic acid; EDC, 1-Ethyl-3-[3-dimethylaminopropyl]carbodiimide Hydrochloride; NHS, N-hydroxysuccinimide; PGA-NHS, N-hydroxysuccinimidyl ester at pending carboxyl groups of PGA; PGA-cystamide, cystamine linked at pending carboxyl groups of PGA via amide bond.

In certain embodiments, it may be is desirable to synthesize a fluorescently labeled conjugate. For example, to synthesize fluorescently labeled PGA-D-pen conjugate, 0.04 ml NHS-fluorescein in DMSO (3.2 mM) may be added to 0.45 ml of PGA-D-pen conjugate in PBS buffer pH 7.4. The reaction mixture may then be stirred in dark for 1 h at room temperature. The fluorescently labeled conjugate may be purified using a Sephadex G-25 column. Moles of fluor per mole of PGA may be determined spectrophotometrically (ε=68000 M⁻¹ cm⁻¹, λₘₐₓ=494 nm).

In certain embodiments the polyglutamic acid-D-pen conjugate can display increased pharmacokinetics as compared to the gelatin-D-pen conjugate. For example, as shown in the below examples, polyglutamic acid-D-pen conjugate can produce a more rapid effect than the gelatin-D-pen conjugate and result in a dose-dependent increase in intracellular ROS leading to cytotoxicity in naïve HL-60 cells within 8 hr.

### V. Cancer Therapies

Compositions of the present invention may be administered to a subject, such as a mammal, a rat, a mouse, a non-human animal, or a human patient, to treat a cancer. Although it is envisioned that the compositions of the present invention may be used to treat virtually any cancer, in certain embodiments, a metal chelator-polymer conjugate may be administered to a subject to treat leukemia, cancer of the lymph node or lymph system, bone cancer, cancer of the mouth and esophagus, stomach cancer, colon cancer, breast cancer, ovarian cancer, a gastric cancer, brain cancer, renal cancer, liver cancer, prostate cancer, melanoma, lung cancer, a tumor, and/or a metastasis

A. Combination Therapies

In order to increase the effectiveness of a metal chelator-polymer conjugate, it may be desirable to combine these compositions and methods of the invention with an agent effective in the treatment of hyperproliferative disease, such as, for example, an anti-cancer agent. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

More generally, such an agent would be provided in a combined amount with an metal chelator-polymer conjugate effective to kill or inhibit proliferation of a cancer cell. This process may involve contacting the cell(s) with an agent(s) and the metal chelator-polymer conjugate at the same time or within a period of time wherein separate administration of the metal chelator-polymer conjugate and an agent to a cell, tissue or organism produces a desired therapeutic benefit. This may be achieved by contacting the cell, tissue or organism with a single composition or pharmacological formulation that includes both a metal chelator-polymer conjugate and one or more agents, or by contacting the cell with two or more distinct compositions or formulations, wherein one composition includes a metal chelator-polymer conjugate and the other includes one or more agents.

The terms “contacted” and “exposed,” when applied to a cell, tissue or organism, are used herein to describe the process by which a therapeutic construct of a metal chelator-polymer conjugate and/or another agent, such as for example a chemotherapeutic or radiotherapeutic agent, are delivered to a target cell, tissue or organism or are placed in direct juxtaposition with the target cell, tissue or organism. To achieve cell killing or stasis, the metal chelator-polymer conjugate and/or additional agent(s) are delivered to one or more cells in a combined amount effective to kill the cell(s) or prevent them from dividing.

The metal chelator-polymer conjugate may precede, be co-current with and/or follow the other agent(s) by intervals ranging from minutes to weeks. In embodiments where the metal chelator-polymer conjugate, and other agent(s) are applied separately to a cell, tissue or organism, one would generally ensure that a significant period of time did not expire between the time of each delivery, such that the metal chelator-polymer conjugate and agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute) as the metal chelator-polymer conjugate. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering the metal chelator-polymer conjugate.

Various combination regimens of the metal chelator-polymer conjugate and one or more agents may be employed. Non-limiting examples of such combinations are shown below, wherein a composition of the metal chelator-polymer conjugate is “A” and an agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the composition of the metal chelator-polymer conjugate to a cell, tissue or organism may follow general protocols for the administration of chemotherapeutics, taking into account the toxicity, if any. It is expected that the treatment cycles would be repeated as necessary. In particular embodiments, it is contemplated that various additional agents may be applied in any combination with the present invention.

1. Chemotherapeutic Agents

The term “chemotherapy” refers to the use of drugs to treat cancer. A “chemotherapeutic agent” is used to connote a compound or composition that is administered in the treatment of cancer. One subtype of chemotherapy known as biochemotherapy involves the combination of a chemotherapy with a biological therapy.

Chemotherapeutic agents include, but are not limited to, 5-fluorouracil, anthocyanin, bleomycin, busulfan, camptothecin, capecitabine, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, idarubicin, ifosfamide, lapatinib, letrozole, mechlorethamine, melphalan, mitomycin, navelbine, nitrosurea, other platinum containing compounds, paclitaxel, parthenolide, plicamycin, a polyphenolic agent derived from nature, procarbazine, raloxifene, tamoxifen, temozolomide (an aqueous form of DTIC), transplatinum, vinblastine, vinorelbine, and methotrexate, vincristine, or any analog or derivative variant of the foregoing. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

2. Radiotherapeutic Agents

Radiotherapeutic agents include radiation and waves that induce DNA damage for example, γ-irradiation, X-rays, proton beam therapies (U.S. Pat. Nos. 5,760,395 and 4,870,287), UV-irradiation, microwaves, electronic emissions, radioisotopes, and the like. Therapy may be achieved by irradiating the localized tumor site with the above described forms of radiations. It is most likely that all of these agents affect a broad range of damage DNA, on the precursors of DNA, the replication and repair of DNA, and the assembly and maintenance of chromosomes.

Radiotherapeutic agents and methods of administration, dosages, etc. are well known to those of skill in the art, and may be combined with the invention in light of the disclosures herein. For example, dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

3. Surgery

Approximately 60% of persons with cancer will undergo surgery of some type, which includes, for example, preventative, diagnostic or staging, curative and palliative surgery. Surgery, and in particular a curative surgery, may be used in conjunction with other therapies, such as the present invention and one or more other agents.

Curative surgery includes resection in which all or part of cancerous tissue is physically removed, excised and/or destroyed. It is further contemplated that surgery may remove, excise or destroy superficial cancers, precancers, or incidental amounts of normal tissue. Treatment by surgery includes for example, tumor resection, laser surgery, cryosurgery, electrosurgery, and miscopically controlled surgery (Mohs' surgery). Tumor resection refers to physical removal of at least part of a tumor. Upon excision of part of all of cancerous cells, tissue, or tumor, a cavity may be formed in the body.

Further treatment of the tumor or area of surgery may be accomplished by perfusion, direct injection or local application of the area with an additional anti-cancer agent. Such treatment may be repeated, for example, about every 1, about every 2, about every 3, about every 4, about every 5, about every 6, or about every 7 days, or about every 1, about every 2, about every 3, about every 4, or about every 5 weeks or about every 1, about every 2, about every 3, about every 4, about every 5, about every 6, about every 7, about every 8, about every 9, about every 10, about every 11, or about every 12 months. These treatments may be of varying dosages as well.

4. Immunotherapeutic Agents

An immunotherapeutic agent generally relies on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (e.g., a chemotherapeutic, a radionuclide, a ricin A chain, a cholera toxin, a pertussis toxin, etc.) and serve merely as a targeting agent. Such antibody conjugates are called immunotoxins, and are well known in the art (see U.S. Pat. No. 5,686,072, U.S. Pat. No. 5,578,706, U.S. Pat. No. 4,792,447, U.S. Pat. No. 5,045,451, U.S. Pat. No. 4,664,911, and U.S. Pat. No. 5,767,072, each incorporated herein by reference). Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

5. Genetic Therapy Agents

A tumor cell resistance to agents, such as chemotherapeutic and radiotherapeutic agents, represents a major problem in clinical oncology. One goal of current cancer research is to find ways to improve the efficacy of one or more anti-cancer agents by combining such an agent with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that gene therapy could be used similarly in conjunction with the metal chelator-polymer conjugate and/or other agents.

### VI. Pharmaceutical Preparations

Pharmaceutical compositions of the present invention comprise an effective amount of one or more metal chelator-polymer conjugate or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one metal chelator-polymer conjugate or additional active ingredient will be known to those of skill in the art in light of the present disclosure, as exemplified by Remington: The Science and Practice of Pharmacy, 21^{st} edition, by University of the Sciences in Philadelphia, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the pharmaceutical compositions is contemplated.

The metal chelator-polymer conjugate may comprise different types of carriers depending on whether it is to be administered in solid, liquid or aerosol form, and whether it need to be sterile for such routes of administration as injection. The present invention can be administered intravenously, intradermally, intracranially, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). In certain embodiments, a metal chelator-polymer conjugate of the present invention is administered intravenously or parenterally.

The metal chelator-polymer conjugate may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include a metal chelator-polymer conjugate, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the metal chelator-polymer conjugate may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the metal chelator-polymer conjugate are formulated to be administered via an alimentary route; for example, this approach may be particularly useful for treating stomach cancer, gastrointestinal cancer, and/or colon cancer. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof, an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, metal chelator-polymer conjugate may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound metal chelator-polymer conjugate may be formulated for administration via various miscellaneous routes, for example, oral, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and laurocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroethylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

### Cell Lines and Culture Conditions

The human breast cancer cell lines BT474 (her2 positive and ER+), and MCF-7 (her2 negative and ER+) and human leukemia cell line, HL-60, were purchased from American Type Cell Culture Collection (ATCC, Rockville, Md.). Resistant leukemia cell lines, HL-60/VCR (P-gp) and HL-60/ADR (MRP-1) were kindly provided by Dr Baer (Roswell Park Cancer Institute, Buffalo, N.Y.). Cells were routinely cultured in RPMI-1640 media (Invitrogen, Carlsbad, Calif.) supplemented with 100 U/mL penicillin, 100 μg/mL, streptomycin and 10% Fetal Bovine Serum (FBS) (ATCC, Rockville, Md.) and maintained at 37° C. in a humidified 5% CO₂ incubator. Plasmocin (5 μg/mL) (InvivoGen, San Diego, Calif.) was added to the cell culture media as a prophylactic measure to prevent mycoplasma contamination. Cell viability was regularly determined by trypan blue exclusion test.

### Reagents

D-penicillamine (D-pen), hydrogen peroxide (H₂O₂) 30% w/w and cupric sulfate (CuSO₄), catalase (2860 U/mg), glutathione, 5,5′-dithiobis (2-nitrobenzoic acid) (DTNB), EDTA, were purchased from Sigma-Aldrich Inc. (St. Louis, Mo.). Coomassie Plus Protein Assay kit was purchased from Pierce Biotech Inc. (Rockford, Ill.). Dimethylsulfoxide (DMSO) was purchased from Fisher Scientific (Pittsburgh, Pa.). Cell cytotoxicity was assessed with the 3-(4,5-dimethylthiazol-2-yl)-2,5-diphenyltetrazolium bromide) (MTT) assay, phosphate buffer saline (PBS), pH 7.4 cell culture grade was purchased from ATCC (Rockville, Md.), 2′-7′-dichlorodihydrofluorescein diacetate (H₂DCFDA), and hydroethidine were purchased from Invitrogen Inc (Carlsbad, Calif.).

### Assessment of Cell Viability

Cell viability was measured using the 3-(4,5-dimethyl-2-yl)-2,5-diphenylteraolium bromide (MTT) assay. The adherent breast cancer cells (MCF-7 and BT474) were seeded at an initial concentration of 3×10⁴ cells/well 24 h prior to the commencement of the experiments to allow them to attach, while the suspension leukemia cells, HL-60, HL-60-/VCR and HL-60/ADR were seeded at 3×10⁴ cells/well on the same day of the experiment in a 96 well plate. Cells were then treated with H₂O₂ (1-200 μM), D-pen (1-400 μM), cupric sulfate (1-50 μM), D-pen (1-400 μM)+cupric sulfate (10 μM), and D-pen (1-400 μM)+cupric sulfate (10 μM)+catalase (500 U/mL). The MTT assay was performed at 48 h and absorbance was measured with the microplate reader (Biotek ELₓ 800, Biotek Instruments, Winooski, Vt.) at a wavelength of 595 nm. Data is reported as cell viability (% control) and corresponds to the percent viable cells compared to untreated cells.

### Determination of Intracellular Reactive Oxygen Species (ROS)

2′-7′-dichlorodihydrofluorescein diacetate (H₂DCFDA) was used as an indicator of intracellular ROS generation. H₂DCFDA is a cell permeable probe, it enters the cell and is deacetylated to a non-fluorescent product, 2′-7′-dichlorodihydrofluorescein (H₂DCF) by cellular esterases and is oxidized by ROS to a fluorescent product, 2′-7′-dichlorofluorescein (DCF). MCF-7 and HL-60 cells were loaded with H₂DCFDA and were seeded in a 96 well plate at a concentration of 3×10⁴ cells/well. Briefly, cells were incubated with PBS buffer containing 5 μM H₂DCFDA (dissolved in DMSO) for 30 min at 37° C. H₂DCFDA was then removed and cells were washed twice with fresh PBS to remove excess H₂DCFDA. Cells were then incubated with either drug in media or media alone (positive control). Non-H₂DCFDA loaded cells were used as negative control. The fluorescence of control and sample wells was recorded at excitation 485±20 nm, emission 530±25 nm with Biotek FL600 (Biotek Instruments, Winooski, Vt.) at 30, 60, and 90 min. Then data is reported as DCF fluorescence (fold increase vs. control) corresponding to the increase of fluorescence associated with the sample cells compared to that of untreated cells.

### Intracellular Levels of Thiols (—SH)

The intracellular reduced glutathione levels were measured with 5,5′-dithiobis-(2-nitrobenzoic acid) (DTNB). Briefly, HL-60 cells (5×10⁶) were seeded in a 12 well plate and incubated for 4 and 48 h with or without D-pen (50 and 200 μM) plus cupric sulfate (10 μM). Cells were collected, washed with ice-cold PBS buffer and then suspended in pH 7.4 lysis buffer comprised of 25 mM Tris-HCl+1 mM EDTA+0.5% Triton X-405. Cells were then lysed in the buffer with a homogenizer (Tissue Tearor, Model 985370 Variable Speed, Biospec Products Inc, Bartlesville, Okla.). 25 μL of standard (glutathione) or samples (cell lysate)+25 μL DTNB (0.4 mg/mL)+150 μL of 50 mM HEPES buffer, pH 7.4 with 5 mM EDTA were incubated for 10 min at room temperature and read at 405 nm on a microplate reader.

### Protein Assay

The total protein content of the cell lysate was analyzed with Coomassie Plus Bradford Protein Assay using Glutathione as a standard.

### Statistical Analysis

Data are represented as the mean±standard error (SE). Each experimental group consisted of n=6 and experiments were repeated two times. Statistical analysis was performed with two-way analysis of variance (ANOVA) followed by Bonferroni post test where significance was set at P<0.05 with GraphPad Prisms 4 Software (GraphPad software Inc. San Diego, Calif.). The IC₅₀ was calculated with GraphPad Prism® non-linear regression program.

### Example 2

### Copper Chelation by D-Penicillamine Generates Reactive Oxygen Species that are Cytotoxic to Human Leukemia and Breast Cancer Cells

The inventors recently investigated the mechanism of copper catalyzed D-pen oxidation and simultaneous H₂O₂ production as a function of time, concentration of cupric sulfate or ferric chloride, temperature, pH, anaerobic conditions, and in the presence of chelators such as EDTA and bathocuproinedisulfonic acid (BCS) [33]. It was demonstrated that H₂O₂ was generated in a concentration dependent manner as a result of D-pen oxidation in the presence of cupric sulfate. Chelators such as EDTA and BCS were able to inhibit D-pen oxidation [33]. Additionally, it was shown that the in-vitro copper catalyzed D-pen oxidation generates H₂O₂ in a 2:1 mole ratio at low D-pen concentrations (<500 μM) [33]. Therefore, the purpose of these studies was to, 1) examine the cytotoxicity due to the ROS generating ability of D-pen, and 2) if the cytotoxicity of D-pen in the presence of copper correlated to the in-vitro non-cell based molar ratio of D-pen and H₂O₂. Breast cancer cell lines differing in her2 expression [MCF-7 (her2 negative) and BT474 (her2 positive)], and leukemia cell lines differing based on their anthracycline sensitivity [HL-60 (wild type), HL-60/VCR (P-gp) and HL-60/ADR (MRP-1)] were used in these studies to ascertain differences in H₂O₂ and ROS cytotoxic effects. “P-gp” refers to P-glycoprotein.

The specific aim of the current study was to assess whether D-pen at low concentration (≦400 μM) in the presence of copper would cause intracellular generation of ROS and result in cytotoxicity in cancer cells. Indeed, in the current studies, it is demonstrated that D-pen generates concentration dependent ROS only in the presence of copper and exhibits concentration dependent cytotoxicity in cancer cells.

### In-Vitro Cytotoxicity of D-Pen Plus Cupric Sulfate and H₂O₂

### Leukemia Cells

HL-60, HL-60/VCR and HL-60/ADR cells were treated with H₂O₂ (1, 10, 25, 50, 100 and 200 μM) and D-pen (1, 50, 100, 150, 200 and 400 μM) plus cupric sulfate (10 μM). Cells were also treated with cupric sulfate alone as control (0.1, 1, 10, 25 and 50 μM). The concentrations of H₂O₂ and D-pen were chosen based on previous in-vitro non-cell based studies (Gupte and Mumper, 2007), where it was determined that at low D-pen concentrations (<500 μM) copper catalyzed D-pen oxidation resulted in H₂O₂ generation in the molar ratio of 2:1 (D-pen: H₂O₂). Therefore, the inventors wanted to compare the cytotoxicity in these present studies using H₂O₂ alone and versus H₂O₂ generated from D-pen plus cupric sulfate wherein the theoretical H₂O₂ generated corresponded to a 2:1 molar ratio of D-pen to H₂O₂.

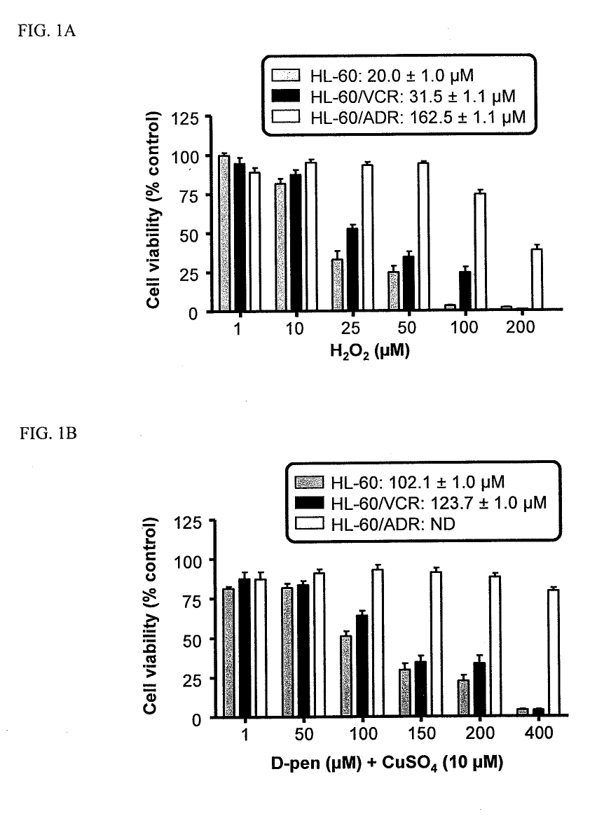

FIGS. 1A-B show the concentration dependent cytotoxicity of H₂O₂ alone (FIG. 1A) and D-pen plus cupric sulfate (FIG. 1B), respectively, in leukemia cells (HL-60, HL-60/VCR and HL-60/ADR). HL-60 and HL-60/VCR cells were highly sensitive to H₂O₂ with IC₅₀ of 20±1.0 μM and 31.5±1.1 μM, respectively. The IC₅₀ of D-pen plus cupric sulfate in these two leukemia cell lines was 102.1±1.0 μM and 123.7±1.0 μM, which was approximately 5-fold and 4-fold more than corresponding IC₅₀ of H₂O₂ alone in these two cell lines. The HL-60/ADR cells were shown to be less sensitive to the cytotoxic effects of both H₂O₂ and D-pen plus cupric sulfate with IC₅₀ of H₂O₂ of 162.5±1.1 μM, while the IC₅₀ of D-pen plus cupric sulfate was beyond the concentration range of D-pen used in the present studies. Cupric sulfate alone (0.1-50 μM) did not result in any appreciable loss in cell viability (data not shown). The purpose of employing the resistant leukemia cells, HL-60/VCR (p-gp) and HL-60/ADR (MRP-1), was to compare the effect of H₂O₂ and D-pen plus cupric sulfate cytotoxicity on these cells versus the HL-60 cells. The order of sensitivity of leukemia cells to both H₂O₂ and D-pen plus cupric sulfate cytotoxicity was HL-60>HL-60/VCR>HL-60/ADR and correlated with the sensitivity of these cells to anti-cancer agents. These results support the relationship between ROS and drug resistance (Pelicano et al., 2004).

### Breast Cancer Cells

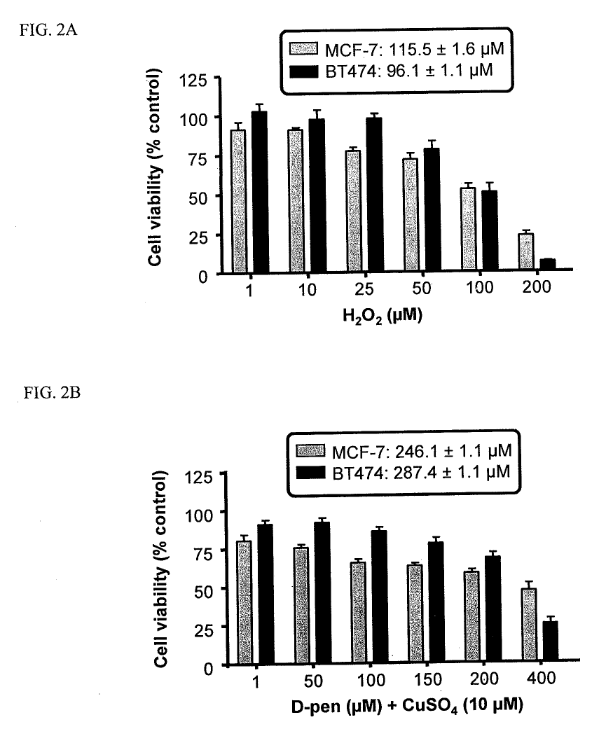

The cytotoxicity of H₂O₂ and D-pen plus cupric sulfate in breast cancer cells is shown in FIGS. 2A-B. The IC₅₀ of H₂O₂ was 115.5±1.6 μM and 96.1±1.1 μM for MCF-7 and BT474 breast cancer cells (FIG. 2A). In comparison, as shown in FIG. 2B, the IC₅₀ of D-pen plus cupric sulfate was 246.1±1.1 μM and 287.4±1.1 μM for MCF-7 and BT474 cells, respectively. The IC₅₀ of H₂O₂ was approximately 2-fold and 3-fold lower than the IC₅₀ of D-pen plus cupric sulfate in MCF-7 and BT474 cells, respectively.

Catalase Protects MCF-7 and HL-60 Cells from D-Pen Plus Cupric Sulfate Cytotoxicity

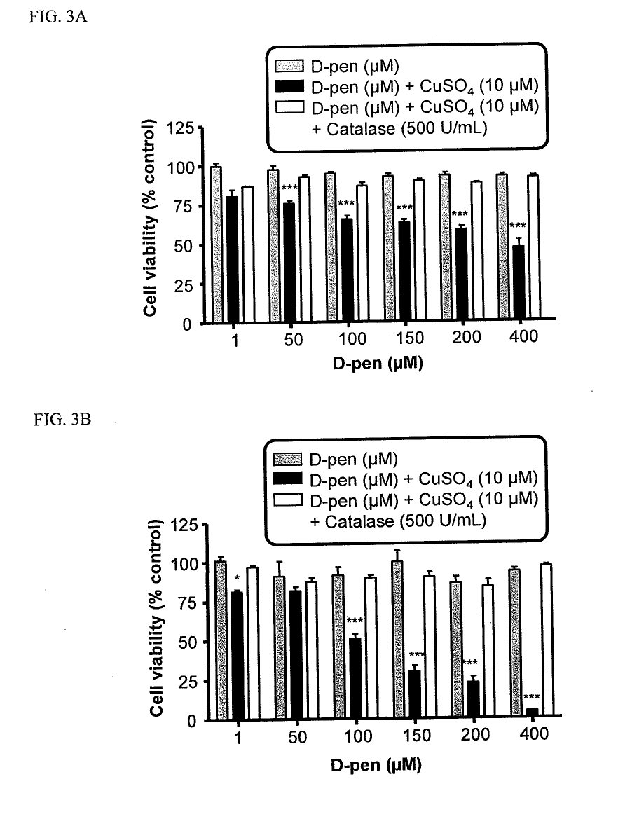

FIGS. 3A-B demonstrates that catalase (500 U/mL) was able to completely protect both MCF-7 (FIG. 3A) and HL-60 cells (FIG. 3B) from D-pen plus cupric sulfate cytotoxicity.

### Intracellular Reactive Oxygen Species (ROS) Generation in MCF-7 and HL-60 Cells

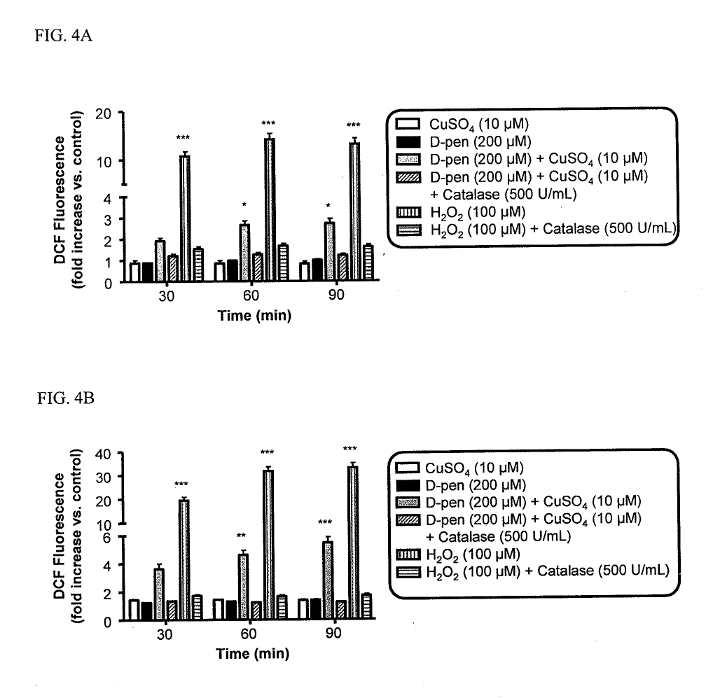

FIGS. 4A-B show that intracellular ROS was produced in both MCF-7 (FIG. 4A) and HL-60 cells (FIG. 4B) in the presence of D-pen (200 μM) plus cupric sulfate (10 μM). D-pen alone (200 μM) or cupric sulfate alone (10 μM) failed to produce any ROS, supporting the fact that copper interaction with D-pen is essential and is responsible for the generation of ROS. Further, a 4-fold increase in ROS production was observed in D-pen plus cupric sulfate treated HL-60 cells versus untreated HL-60 control cells compared to a 2-fold increase in ROS production for D-pen plus cupric sulfate treatment of MCF-7 versus untreated MCF-7 control cells. In addition, the presence of catalase (500 U/mL) with D-pen plus cupric sulfate completely inhibited ROS generation, demonstrating that H₂O₂ was the major ROS generated. As shown in FIGS. 4A-B, when H₂O₂ alone (100 μM) was incubated with HL-60 and MCF-7 cells, 5-fold greater ROS was generated in each cell line (compared to D-pen plus cupric sulfate) which agrees very well with the 5-fold reduced IC₅₀ calculated for H₂O₂ compared to D-pen plus cupric sulfate in HL-60 cells.

In separate cell studies, hydroethidine was used as a quantitative marker for intracellular superoxide anion production. Hydroethidine is freely permeable into cells and can be directly oxidized to a fluorescent compound by intracellular superoxide anion. These studies showed that there was no statistical difference in the intracellular superoxide anion at up to 90 min post-incubation between all treatment and control groups, suggesting that the observed cytotoxicity was not caused by superoxide anion but by hydrogen peroxide.

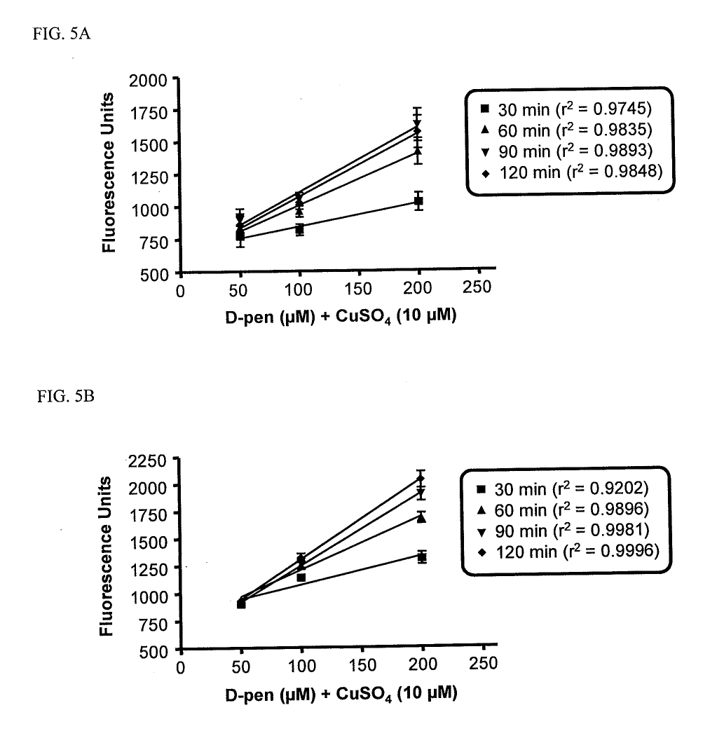

FIGS. 5A-B shows the linear relationship between D-pen (50, 100 and 200 μM) in the presence of cupric sulfate and the increase in the DCF fluorescence (indicator of intracellular ROS) in both MCF-7 (FIG. 5A) and HL-60 (FIG. 5B). The correlation between D-pen concentration and DCF fluorescence was shown to increase over time, as indicated by the r² values becoming higher over time and closer to r²=1.

### Intracellular Reduced Thiols

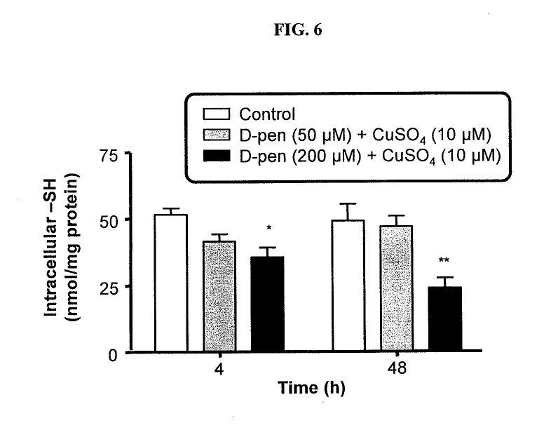

FIG. 6 shows the levels of intracellular thiols (mainly glutathione) in HL-60 cells after incubation with D-pen (50 and 200 μM) plus cupric sulfate (10 μM) for 4 and 48 h. The levels of reduced thiols was shown to be significantly decreased (P<0.05) after incubation with D-pen (200 μM) plus cupric sulfate compared to control at 4 h. After 48 h incubation, intracellular thiol levels were significantly lower (P<0.01) after D-pen plus cupric sulfate incubation compared to control.

### Discussion

Starkebaum et al. (1985) proposed a free radical mechanism of copper catalyzed D-pen oxidation to D-pen disulfide and the subsequent generation of H₂O₂. The mechanism involves an initial reduction of Cu(II) to Cu(I) by D-pen. This is followed by the generation of superoxide anion and finally of H₂O₂ during the spontaneous oxidation of Cu(I) to Cu(II). The inventors have recently further investigated the mechanism proposed by Starkebaum et al. (1985) as a function of time, concentration of cupric sulfate or ferric chloride, temperature, pH, anaerobic conditions and in the presence of chelators (Gupte and Mumper, 2007). It was shown with a HPLC assay that H₂O₂ was indeed generated in a concentration dependent fashion as a result of D-pen oxidation in the presence of cupric sulfate.

In the present studies, it was hypothesized that D-pen at low concentrations (≦400 μM) in the presence of cupric sulfate would generate cellular H₂O₂ and ROS and result in cytotoxicity. The anthracycline sensitive HL-60 and the mildly resistance HL-60/VCR leukemia cells were found to be highly susceptible to D-pen plus cupric sulfate cytotoxicity. The IC₅₀ of H₂O₂ alone was approximately 5-fold and 4-fold lower compared to D-pen plus cupric sulfate in HL-60 and HL-60/VCR cells, respectively. The ROS assay showed that approximately 5-fold higher cellular ROS was generated in HL-60 cells due to incubation with H₂O₂ alone compared to D-pen plus cupric sulfate, which was remarkably similar to the measured 5-fold higher cytotoxicity with H₂O₂ alone compared to D-pen. H₂O₂ was also established to be the major ROS species, as the presence of catalase completely inhibited D-pen plus cupric sulfate cytotoxicity. Breast cancer cells (MCF-7 and BT474) were less sensitive to both H₂O₂ and D-pen cytotoxicity, with 5-fold higher IC₅₀ compared to leukemia cells. The IC₅₀ of H₂O₂ was approximately 2-fold and 3-fold lower compared to D-pen plus cupric sulfate in MCF-7 and BT474 cells, respectively.

In conclusion, these studies demonstrated that low concentration of D-pen (≦400 μM) in the presence copper resulted in a concentration dependent H₂O₂-mediated cytotoxicity in both breast cancer and leukemia cells. D-pen in the presence of copper also was shown to generate concentration dependent cellular ROS and to decrease cellular reduced thiol content in cancer cells. Further, it was shown that leukemia cells were highly sensitive to D-pen plus cupric sulfate cytotoxicity. A four-fold increase in ROS generation was shown in leukemia cells after D-pen plus cupric sulfate treatment compared to untreated control. It was demonstrated that D-pen has effective ROS generating ability in the presence of copper. Since copper levels are significantly elevated in the serum and tumor tissue in a variety of malignancies, these findings provides a novel and exciting opportunity to exploit D-pen as an anti-cancer agent having both anti-angiogenic and cytotoxic mechanism of action.

### Example 3

### Synthesis of a Novel Gelatin-D-Penicillamine Conjugate for the Intracellular Delivery of the Copper Chelator as a Potential Anti-Cancer Agent

### Materials

Type B gelatin (75 bloomstrength) with 100-115 mmol of carboxylic acid per 100 g of protein, an isoelectric point of 4.7-5.2, and an average molecular weight of 20,000-25,000 Da, D-penicillamine (D-pen), D-penicillamine disulfide, Glutathione, Dithiothreitol (DTT) were purchased from Sigma-Aldrich Chemical Co. (St. Louis, Mo.). Sulfosuccinimidyl 6-[3′(2-pyridyldithio)-propionamido]hexanoate (Sulfo-LC-SPDP), 2,4,6-trinitrobenzene sulfonic acid (TNBS) reagent, N-hydroxysuccinimide-Fluorescein (NHS-Fluorescein), M-PER mammalian cell extraction reagent were purchased from Pierce Biotech Inc. (Rockford, Ill.). Fluorescein standard was purchased from Invitrogen Inc. (Carlsbad, Calif.). Acetonitrile, o-phosphoric acid (85%), Falcon 75 cm² polystyrene culture flasks (tissue culture treated, 0.2 μm vented cap, canted neck) and Falcon 96, 48, 6-well polystyrene plates (tissue culture treated, flat bottom, low evaporation lids) were purchased from Fisher Scientific (Pittsburgh, Pa.). Microcon (YM-10, MWCO: 10 kDa) centrifugal filter devices were purchased from Millipore (Billerica, Mass.). All aqueous solutions were prepared in deionized distilled water (MilliQ, Millipore Inc.).

### Synthesis and Purification of the Gelatin-D-Pen Conjugate.

A heterobifunctional cross-linker (Sulfo-SPDP) was employed to conjugate D-pen with gelatin. Sulfo-SPDP has an amine reactive N-hydroxysuccinimide (NHS) and a thiol reactive 2-pyridylthio group. The synthesis was performed in two steps. Briefly, gelatin (10 mg/mL) was incubated with increasing amounts of SPDP for 2 h at room temperature in PBS, pH 7.4. Excess SPDP was separated from gelatin with microcon centrifugation devices (MWCO: 10 kDa) as described below. Pyridine-2-thione and the TNBS assay described below were performed to determine the degree of SPDP modification of gelatin. The SPDP-modified gelatin was then incubated with D-pen. The gelatin-D-pen conjugate was separated from the unconjugated D-pen with microcon centrifugal devices as described below. The HPLC assay described below was performed on the filtrate and retentate to determine the amount of D-pen conjugated to gelatin.

The complete and efficient separation of gelatin from unreacted SPDP or D-pen was performed with microcon centrifugation tubes (MWCO: 10 kDa). Briefly, 500 μL of reaction mixture was added to the microcon tubes, the mixture was centrifuged at 14,000 g for 30 min at room temperature. The filtrate was collected and 400 μL of fresh PBS buffer was added to the retentate. The centrifugation was performed again at 14,000 g at 30 min. Filtrate was collected and the retentate was then collected by centrifuging at 3,000 g for 5 min. Both the filtrate and the retentate were then analyzed. Gelatin was detected and quantitated with coomassie, TNBS and the pyridine 2-thione assay. D-pen was detected and with either the HPLC assay.

Determination of Gelatin. Gelatin concentration was analyzed with Coomassie Protein assay reagent. Briefly, 10 μL of standard or sample was added to 150 μL of Coomassie® Plus Protein Assay Reagent (Pierce Biotech, Rockford, Ill.), mixed and incubated for 10 min at room temperature and the absorbance was read at 595 nm with the Synergy™ 2 multi-detection microplate reader (Biotek, Winooski, Vt.).

Determination of the Level of SPDP modification of Gelatin. The degree of SPDP modification of gelatin was determined by quantifying the release of pyridine-2-thione group after exposure of the SPDP-modified gelatin with DTT. Briefly, 100 μL desalted SPDP-modified gelatin was diluted to 1 mL with PBS buffer, pH 7.4. Ten (10) μL DTT (15 mg/mL) was added and samples were incubated for exactly 15 min and the absorbance was recorded at 343 nm with the Synergy™ 2 multi-detection microplate reader (Biotek, Winooski, Vt.). The molar ratio of SPDP to gelatin modification as follows=(ΔA×8080)×(Average m.w. of gelatin×mg/mL of gelatin). Where the value 8080 reflects the extinction coefficient for pyridine-2-thione at 343 nm: 8.08×10³ M⁻¹ cm⁻¹.

Determination of D-pen. D-pen content was analyzed with a previously developed rapid, sensitive HPLC method. Briefly, a HPLC system [Finnigan™ Surveyor System (Thermo Electron Corp. San Jose, Calif.)] was used and the data was analyzed with the ChromQuest™ software version. 4.2. The mobile phase employed was a 50%-50% v/v mixture of solvent A (50 mM phosphoric acid) and solvent B (50 mM phosphoric acid+5% acetonitrile), both adjusted to pH 2.5, pumped at a flow rate of 1 mL/min. D-pen was detected by UV absorption at 214 nm with retention time of 3.1±0.01 min. Sample concentrations (μM) were obtained from the regression line of peak area versus standard sample concentration (μM). These were calculated using a ten-point calibration curve of D-pen dissolved in PBS buffer, pH 7.4.

D-pen release from the Gelatin-D-pen Conjugate in presence of Glutathione. To evaluate the amount of D-pen released from the conjugate in simulated intracellular reducing conditions, the gelatin-D-pen conjugate was incubated in PBS buffer, pH 6.2 and 7.4 with increasing concentrations of glutathione (0, 0.1, 1 and 10 mM) for 2 h at 37° C. Additionally, D-pen release from the conjugate at various time points was also determined in PBS at pH 6.2 and 7.4 after incubation with glutathione (1 mM) at 37° C.

### Example 4

### In Vitro Evaluation of a Novel Gelatin-D-Penicillamine Conjugate for the Intracellular Delivery of the Copper Chelator as a Potential Anti-Cancer Agent

The overall objectives of this study were: 1) to synthesize and characterize a novel gelatin-D-pen conjugate, 2) to evaluate the release of D-pen from the gelatin-D-pen conjugate in presence of glutathione, 3) to evaluate the in-vitro cytotoxicity and intracellular uptake of D-pen in naïve and copper pre-treated human leukemia cells (HL-60).

### Cell Lines and Culture Conditions.

The human leukemia cell line (HL-60) was purchased from American Type Cell Culture Collection (ATCC, Rockville, Md.). Cells were routinely cultured in RPMI-1640 media (Invitrogen, Carlsbad, Calif.) supplemented with 100 U/mL penicillin, 100 μg/mL streptomycin and 10% Fetal Bovine Serum (FBS) (ATCC, Rockville, Md.) and maintained at 37° C. in a humidified 5% CO₂ incubator. Plasmocin (5 μg/mL) (InvivoGen, San Diego, Calif.) was added to the cell culture media as a prophylactic measure to prevent mycoplasma contamination. Cell viability was regularly determined by trypan blue dye (0.4% in phosphate buffered saline) (ATCC, Rockville, Md.).

### Fluorescence Labeling of the Gelatin-D-Pen Conjugate.

The fluorescent labeling of the conjugate was performed with the aid of an amine reactive fluorescent probe, NHS-Fluorescein. Fluorescein was labeled to either gelatin or D-pen to synthesize Fluorescein-gelatin-D-pen and gelatin-D-pen-Fluorescein conjugate. Briefly, gelatin was incubated with equal molar concentration of NHS-fluorescein for 1 h at room temperature to form the fluorescein labeled gelatin. Microcon® centrifugal filter devices (Ultracel YM-10; 10,000 MWCO) (Millipore Corp., Billerica, Mass.) were used to separate the unreacted fluorescein. D-pen was then conjugated to the fluorescein labeled gelatin as described above. To label D-pen with fluorescein, after the gelatin-D-pen has been synthesized, equal molar concentrations of fluorescein was added to the conjugate, followed by separating the unreacted free fluorescein with centrifugation.

### In-Vitro Characterization of the Gelatin-D-Pen Conjugate.

Determination of Amino group content of Gelatin. 2,4,6-trinitrobenezene sulfonic acid (TNBS) is a rapid and sensitive assay reagent employed for the determination of the free amino groups. Briefly, 50 μL of 0.01% (w/v) TNBS was added to 100 μL of either gelatin or SPDP-modified gelatin in PBS buffer, pH 7.4 and was incubated for 2 h at 37° C. Twenty five (25) μL of 1 N HCl was added and the absorbance was recorded at 335 nm. A standard curve was generated using known concentrations of D-pen. The primary amino group was expressed as the amount of TNBS reactive amino groups in 1 g of gelatin. The % amino group conjugated was determined as follows: [Amino group of SPDP modified gelatin/amino groups in gelatin alone]×100.

### In-Vitro Cell Uptake Studies

Quantitative Analysis of Fluorescein labeled Gelatin-D-pen Conjugate Association with HL-60 cells. The HL-60 cells were seeded in a 24 well plate at a density of 10⁵ cells/mL in 1 mL of medium containing either fluorescein alone, fluorescein labeled gelatin, fluorescein-gelatin-D-pen and gelatin-D-pen-fluorescein conjugate. At predetermined time points of 4, 24, 48, and 72 h cells were transferred to a centrifuge tube, washed twice with fresh PBS. Cells were lysed with M-PER mammalian cell extraction reagent and the cellular associated fluorescence was determined. The amount of fluorescence associated with the cells was measured by quantifying the intensity of fluorescence at 485±20 nm (excitation) and 528±20 nm (emission), respectively with the Synergy™ 2 multi-detection microplate reader (Biotek, Winooski, Vt.). Total cellular protein content was determined with Coomassie assay. The percent of gelatin-D-pen conjugate cell association was calculated from the ratio of the observed cell associated fluorescence to the total fluorescence added to the cells.

Quantitative cell uptake of free D-pen. The quantitative cell uptake of free D-pen was investigated in HL-60 cells. Briefly, D-pen (100 μM) was incubated with HL-60 (1×10⁶) cells in PBS, pH 7.4 at 37° C. in a 5% CO₂ incubator. Cells were incubated for 1-4 h. At pre-determined time the cells were separated by centrifugation and the supernatant was analyzed for the remaining concentration of D-pen as present as either free D-pen or D-pen disulfide using the HPLC assay previously described (Gupte and Mumper, 2007).

Qualitative Analysis of Fluorescein labeled Gelatin-D-pen Conjugate Uptake by HL-60 cells. HL-60 cells were seeded at the density of 10⁵ cells/mL in a 96 well plate. 10 μL of fluorescein alone, or fluorescein labeled gelatin alone or fluorescein labeled gelatin-D-pen conjugate was added to the cell suspension. At predetermined time points of 4, 24, 48 72, and 96 h cells were transferred to a centrifuge tube, washed twice with PBS. Cells were transferred onto a slide for visualizing using an Olympus fluorescence microscope.

In-vitro cytotoxicity of gelatin-D-pen conjugate. The HL-60 cells were seeded at a density of 2×10⁴ cells/mL in a 96 well plate. The in-vitro cytotoxicity of the gelatin-D-pen conjugate was determined as follows: first, HL-60 cells were incubated with D-pen (0.1-500 μM), gelatin (0.1-1000 μg/mL), gelatin-D-pen conjugate either alone or with cupric sulfate (10 μM). Next, HL-60 cells were pre-treated with cupric sulfate (0.1 and 10 μM) for 96 h. The above experiments were repeated with the copper pre-treated cells. Cell viability was determined at 24 and 72 h with MTT assay.

The temperature dependent cellular association of the fluorescein alone, fluorescein-gelatin, fluorescein-gelatin-D-pen, gelatin-D-pen-fluorescein conjugate was compared by the following method. The conjugates were incubated at 37° C. and 4° C. for 1 h with HL-60 cells. After the incubation, cell-associated fluorescence is measured at 485±20 nm (excitation) and 528±20 nm (emission). Fluorescein labeling of gelatin was ˜0.183-0.27 moles of fluor/mole of gelatin.

The intracellular accumulation of fluorescein alone, fluorescein-gelatin, fluorescein-gelatin-D-pen and gelatin-D-pen-fluorescein-conjugate was evaluated in HL-60 human leukemia cells. Differential interference contrast (DIC) and fluorescence microscopy images of the cells were obtained after 4, 24, 48 and 72 h.

Copper pre-treatment did not result in statistically significant decrease in cell number or viability, even at cupric sulfate concentration up to 100 μM (p>0.05 for all the cupric sulfate treatment cells compared to control (cupric sulfate: 0 μM).

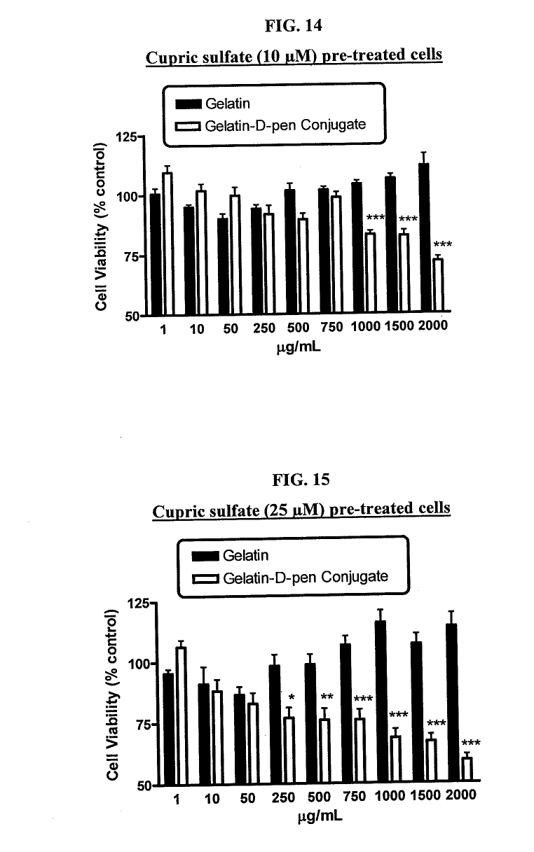

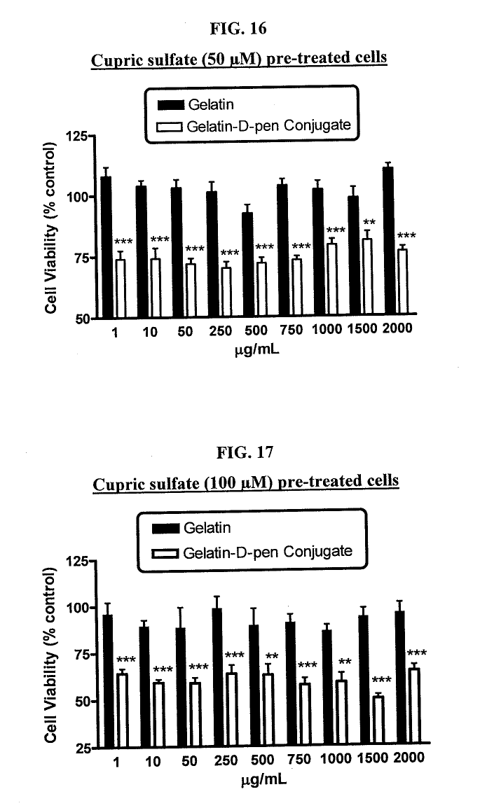

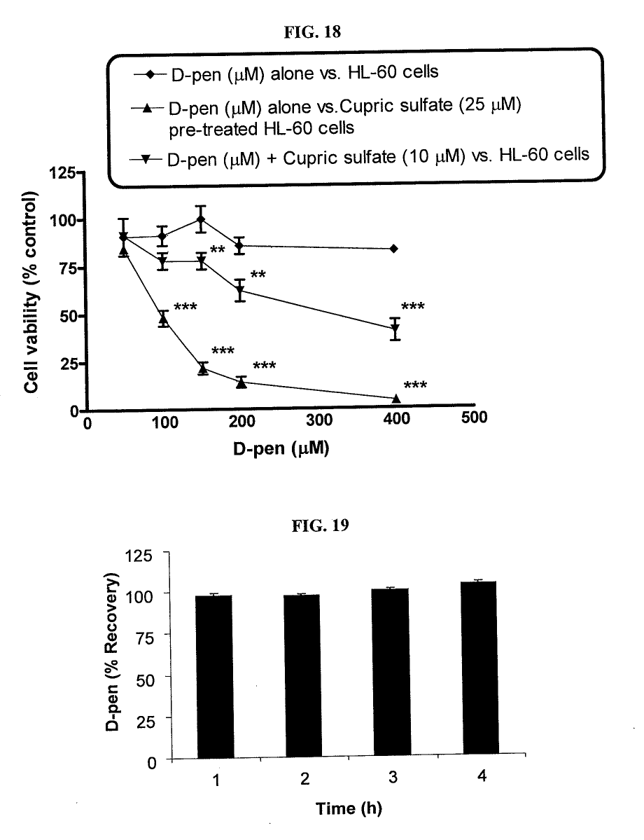

Gelatin-D-pen resulted in a statistically significant reduction in cell viability of cells pretreated with cupric sulfate, as shown in FIG. 13, FIG. 14, FIG. 15, FIG. 16, FIG. 17. The potency of gelatin-D-pen was particularly notable, with substantial decreases in cell viability being observed at 1 μg/ml.

### Example 5

### Gelatin-D-Pen Pharmacology

D-penicillamine (D-pen) is an established copper chelator. The copper catalyzed D-pen oxidation generates concentration dependent hydrogen peroxide (H₂O₂). Additionally, D-pen co-incubated with cupric sulfate resulted in cytotoxicity in human leukemia and breast cancer cells due to the extracellular generation of reactive oxygen species (ROS). The inherent physicochemical properties of D-pen such as its short in-vivo half life, low partition coefficient and rapid metal catalyzed oxidation limit its intracellular uptake and the potential utility as an anti-cancer agent in-vivo. Therefore, to enhance the intracellular delivery and to protect the thiol moiety of D-pen, the inventors designed, synthesized, and evaluated a novel gelatin-D-pen conjugate. D-pen was covalently coupled to gelatin with a biologically reversible disulfide bond with the aid of a heterobifunctional crosslinker, (N-succinimidyl-3-(2-pyridyldithio)-propionate) (SPDP). Additionally, fluorescein labeled gelatin-D-pen conjugate was synthesized for cell uptake studies. D-pen alone was shown not to enter leukemia cells. In contrast, the qualitative intracellular uptake of the conjugate in human leukemia cells (HL-60) was shown with confocal microscopy. The conjugate exhibited slow cell uptake (over the period of 48 to 72 h). A novel HPLC assay was developed to simultaneously quantify both D-pen and glutathione in a single run. The conjugate was shown to completely release D-pen in the presence of glutathione (1 mM) in approximately 3 h in PBS buffer, pH 7.4. The gelatin-D-pen conjugate resulted in significantly greater cytotoxicity compared to free D-pen, gelatin alone and a physical mixture of gelatin and D-pen in human leukemia cells. Further studies are warranted to assess the potential of D-pen conjugate in the delivery of D-pen as a ROS generating anti-cancer agent.

Synthesis of gelatin-D-pen conjugate. The gelatin-D-pen conjugate was prepared as described above by a simple two step reaction: i) gelatin was modified with the heterobifunctional cross-linker (sulfo-LC-SPDP); ii) D-pen was conjugated to the modified gelatin (SPDP-gelatin) with simple thiol exchange. The choice of the amine-thiol reactive heterobifunctional cross-linker (sulfo-LC-SPDP) was based on two important properties: i) availability of amino groups in gelatin for chemical modification, ii) the protection of the thiol group of D-pen, through a reversible bond between polymer (gelatin) and the drug (D-pen). A disulfide bond compared to the irreversible (maleimide) bond provides potential biological reversibility.

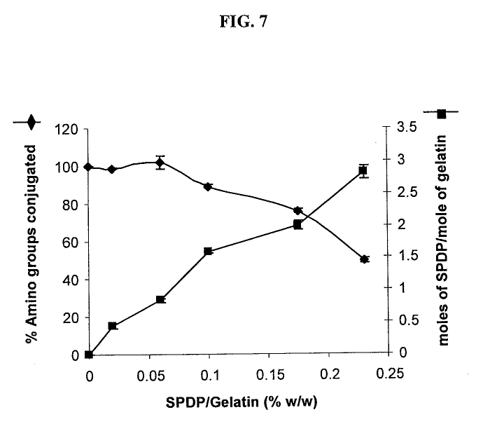

As shown in FIG. 7, the amino group content of gelatin was reduced to approximately 50% of the original gelatin as the ratio of SPDP added to gelatin increased to 0.23% w/w. To confirm and quantify the amount of SPDP modification of gelatin in the process of loss of amino groups, the SPDP modified gelatin was incubated with DTT to release the pyridine-2-thione group. FIG. 7 shows that approximately 3 moles of SPDP were conjugated per mole of gelatin as SPDP added to gelatin increased to 0.23% w/w. This corresponded to 20.5±0.5 to 102.4±1.4 μmol D-pen/g gelatin or 3.1±0.07 to 15.2±0.2 mg D-pen/g gelatin as the ratio of SPDP to gelatin was increased from 0.02-0.23% w/w.

Synthesis of fluorescein labeled gelatin-D-pen conjugate. A fluorescein-gelatin-D-pen conjugate was synthesized for cell uptake studies. The degree of labeling of fluorescein on gelatin was 0.1 mole of fluorescein per mole of gelatin. Gelatin was labeled with fluorescein through the reaction of the NHS-Fluorescein with gelatin to form fluorescein-labeled gelatin. The fluorescein-gelatin was then conjugated to D-pen as described above to form the fluorescein-gelatin-D-pen conjugate. Fluorescein-gelatin alone was used as a control for cell uptake studies.

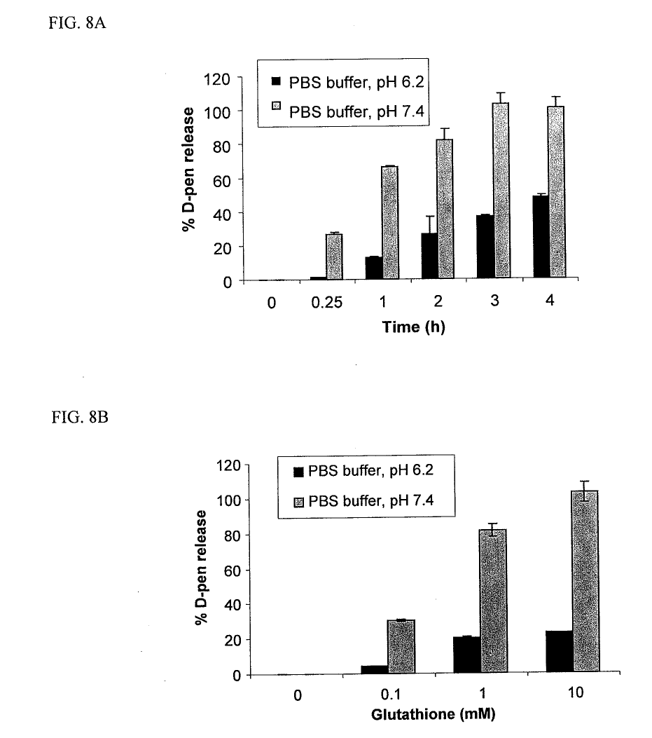

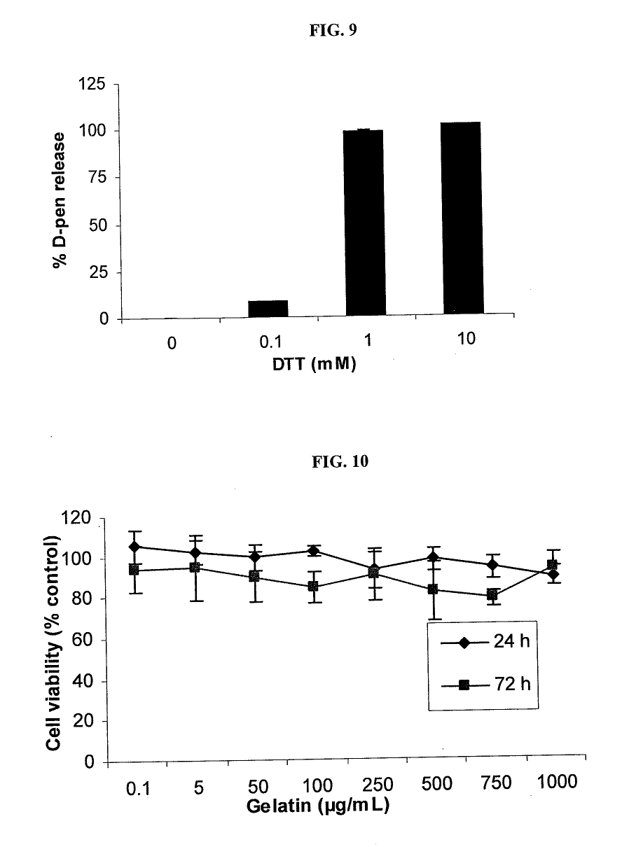

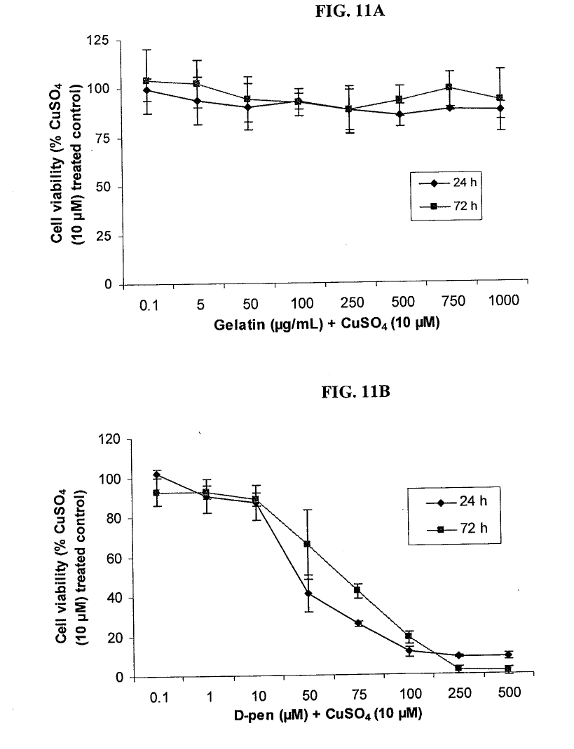

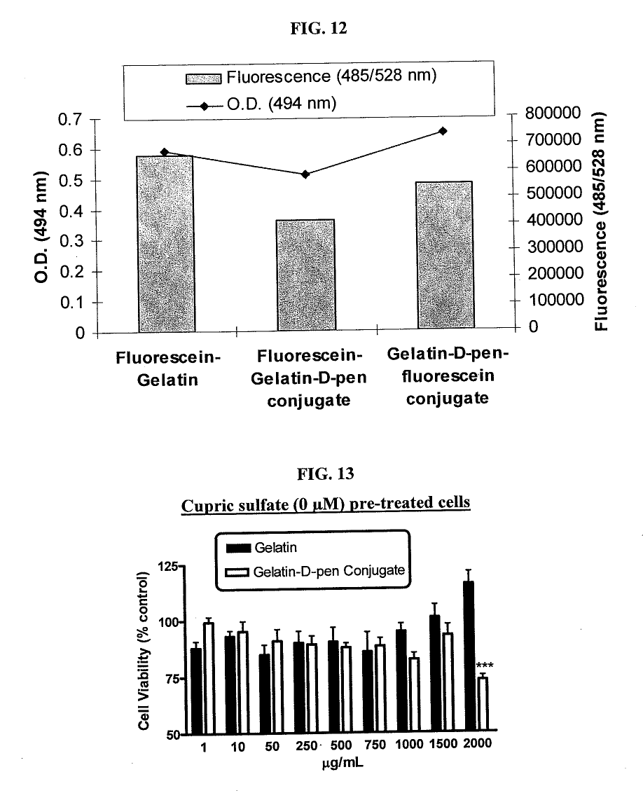

D-pen release in the presence of glutathione. The stability of the disulfide bond between D-pen and gelatin was investigated using glutathione, an endogenous reducing agent. The intracellular glutathione concentration has been reported to range from 1-11 mM (Schafer and Buettner, 2001). Therefore, in-vitro release studies were performed in the presence of both low (1 mM) and high (10 mM) concentrations of glutathione. The pH 6.2 and 7.4 were based on the reported pH of the early endosome and cytosol, respectively. In the presence of 1 mM glutathione in pH 7.4, D-pen was completely released in 4 h, while only ˜50% D-pen was released at pH 6.2 (FIG. 8A). As shown in FIG. 8B, 2 h incubation of the conjugate with glutathione (0.1-10 mM) at pH 7.4 resulted in the release of approximately 30%, 80% and 100% of D-pen in the presence of 0.1, 1, and 10 mM glutathione, respectively. In contrast, the D-pen release at pH 6.2 was approximately 5%, 20% in the presence of 0.1, 1, and 10 mM glutathione, respectively. D-pen and glutathione were simultaneously quantitated as shown using HPLC analysis. These studies underline the significance of interaction of the conjugate at favorable pH and local concentration of glutathione for successful D-pen release and the subsequent cytotoxicity.

Intracellular uptake of free D-pen. Lodemann et al. reported the inability of D-pen to cross cell membrane of mammalian cells (Lodemann, 1981). The inability to be transported is likely explained by the three highly ionizable functional groups of D-pen. The novel HPLC assay developed provides accurate determination of both D-pen and D-pen disulfide. When the supernatant was analyzed for remaining D-pen in the supernatant at 1, 2, 3 and 4 h post-incubation, approximately 100% D-pen was recovered. These results support the previously reported studies by Lodermann et al. (Lodemann, 1981) and signify the need for developing a novel delivery system for D-pen to enhance intracellular delivery.

Gelatin-D-pen conjugate uptake. The intracellular uptake of the gelatin-D-pen conjugate and fluorescein-labeled gelatin alone in HL-60 cells was studied using confocal laser scanning microscopy. Intracellular uptake of gelatin-D-pen conjugate was performed as follows. HL-60 cells were incubated with the fluorescein labeled gelatin-D-pen conjugate at 37° C. for a) 4 and b) 72 h. Cells were washed twice with PBS and cells then suspended in phenol red free RPMI media. Cells were imaged by Confocal Laser Scanning Microscopy. Fluorescence, Differential Contrast (DIC) and overlapped images.

The fluorescein-gelatin-D-pen conjugate uptake into HL-60 cells was a slow process with maximum uptake seen at the 72 h compared to 4 h. Similar slow cell uptake was seen with the fluorescein labeled gelatin. Both the conjugate and gelatin alone were seen to accumulate in the cell in a uniform, non-punctate manner. However, there was a trend for increases fluorescence associated with the cell membrane as compared to the cytoplasm. There were no apparent differences between the cellular uptake of gelatin and the gelatin-D-pen conjugate. Ofner et al. previously showed a similar cellular distribution and slow uptake (over 96 h) of fluorescein-labeled gelatin in HL-60 cells (Ofner et al., 2006). The cell uptake of the conjugate by measuring the cell lysate fluorescence of the conjugate treated HL-60 cells was confirmed. In the below example, the cellular uptake profiles of other polymers such as polyglutamic acid are examined to further improve intracellular D-pen delivery. The below example provides data that the uptake of the polyglutamic acid-D-pen conjugate is much more rapid than that of gelatin-D-pen conjugate and results in dose-dependent increase in intracellular ROS leading to cytotoxicity in naïve HL-60 cells within 8 hr. Moreover, the levels of ROS produced by gelatin-D-pen conjugate (equivalent to 500 μM D-pen dose) is comparable to that caused by 100 μM H₂O₂ whereas free D-pen causes no increase in intracellular ROS since it is cell impermeable.

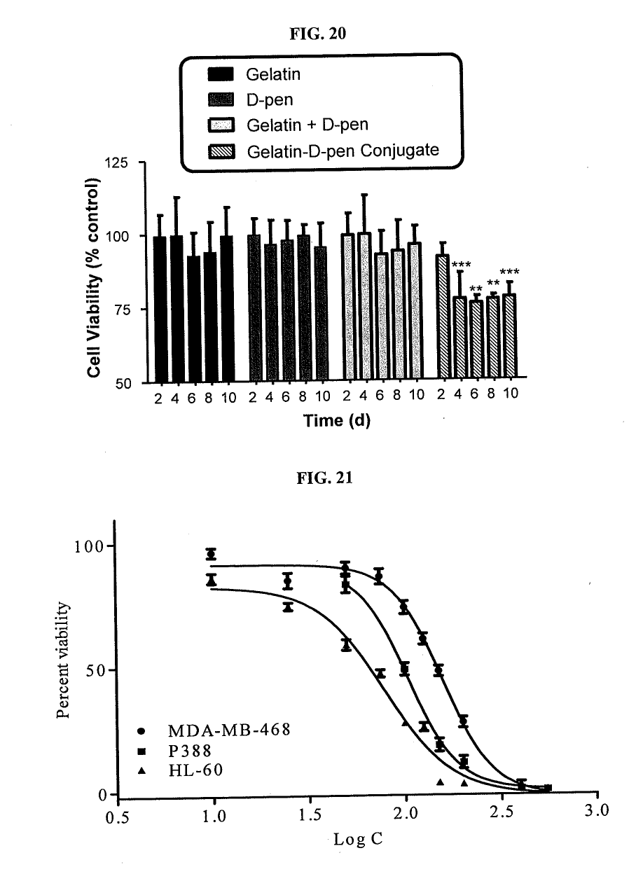

Conjugate cytotoxicity in HL-60 cells. D-pen alone in the presence of cupric sulfate was more cytotoxic in leukemia cells compared to breast cancer cells (Gupte and Mumper, 2007). The data suggest that this may be due to the higher innate copper levels in the cultured leukemia cells compared to breast cancer cells. Therefore, the cytotoxicity of the conjugate was evaluated in human HL-60 leukemia cells. HL-60 cells were treated with gelatin and D-pen alone, a physical mixture of gelatin plus D-pen and the gelatin-D-pen conjugate. As the conjugate was shown to enter the cells, the differences in cytotoxicity of the conjugate was compared with the free gelatin and D-pen and the physical mixture of gelatin plus D-pen in the presence of innate levels of intracellular copper in HL-60 cells. During the studies, media was replaced every two days. As shown in FIG. 20, the conjugate exhibited significantly increased cytotoxicity compared to all the controls (p<0.001 on day 4 and 10 and p<0.01 on day 6 and 8, respectively). The mechanism of D-pen cytotoxicity is due to the generation of H₂O₂ and other ROS in presence of copper (Gupte and Mumper, 2007a; Gupte and Mumper, 2007b). Thus, the intracellular efficacy of D-pen is dependent upon two important processes, namely, i) the interaction of the conjugate with glutathione and the subsequent release of D-pen and ii) the interaction of the released D-pen with the intracellular innate copper present in leukemia cells to produce its cytotoxic effect. Additional studies are ongoing regarding the intracellular D-pen release from the conjugate and the localization of intracellular copper to improve the conjugate anti-cancer effect. Additional conjugates are being designed and synthesized that may increase the rate and extent of uptake and subsequent D-pen release.

In conclusion, a novel method for the synthesis and characterization of a novel gelatin-D-pen conjugate is described. The disulfide bond between gelatin and D-pen in the conjugate provides protection of D-pen from oxidation. Additionally, the disulfide bond in the conjugate was shown to be biologically reversible through the complete release of D-pen only in the presence of biologically relevant concentration of glutathione. It was shown that free D-pen does not enter cells. The delivery of D-pen as a novel gelatin-D-pen conjugate was shown to increase the intracellular accumulation of conjugate in cancer cells over time, and this directly led to enhanced cytotoxicity in human leukemia cells. The cytotoxicity was of the conjugate in leukemia cells was sustained and significant compared to the physical mixture of gelatin and D-pen indicating to the importance of disulfide bond between gelatin and D-pen. To further improve the conjugate cytotoxicity we are currently examining the intracellular interaction of the D-pen released from the conjugate with the cellular copper either free or bound to proteins.

### Example 6

### Poly-l-Glutamic Acid-D-Penicillamine Conjugate for Intracellular Delivery of D-Penicillamine

Materials: Poly-l-glutamic acid sodium salt (MW 20-40 KDa), D-penicillamine (D-pen), D-penicillamine disulfide, Tris(2-carboxyethyl)phosphine hydrochloride (TCEP), 1-Ethyl-3-[3-dimethylaminopropyl]carbodiimide Hydrochloride (EDC), cystamine dihydrochloride, N-hydroxysuccinimide (NHS), Sephadex® G-25 medium and ammonium dihydrogen phosphate were purchased from Sigma-Aldrich. NHS-fluorescein and BCA protein assay kit was purchased from Pierce Biotech Inc. (Rockford, Ill.). Acetonitrile, N,N-Dimethylformamide (DMF), Dimethylsulfoxide (DMSO) and o-phosphoric acid (85%) were purchased from Fisher Scientific (Pittsburgh, Pa.). Carboxy-H2DCFDA was purchased from Invitrogen.

Cell Lines and Culture Conditions. The HL-60 and MDA-MB-468 cells were obtained from American Type Cell Culture Collection (ATCC, Rockville, Md.). The P388 cells were obtained from National Cancer Institute-Frederick Cancer Research Facility, DCT Tumor Repository (NCI, Bethesda, Md.). HL-60 and P388 cells were cultured in RPMI-1640 (Invitrogen, Carlsbad, Calif.) while MDA-MB-468 cells were cultured in DMEM (Invitrogen, Carlsbad, Calif.). The media were supplemented with 100 U/mL penicillin, 100 μg/mL streptomycin and 10% Fetal Bovine Serum (FBS) (ATCC, Rockville, Md.). All cell lines were maintained at 37° C. in a humidified 5% CO₂ incubator. Cell viability was regularly determined by trypan blue dye (0.4% in phosphate buffered saline) (ATCC, Rockville, Md.).

Synthesis of PGA-D-pen Conjugate: PGA-D-pen conjugate was synthesized as shown in FIG. 1. In the first step, cystamine was covalently conjugated to PGA to form PGA-cystamide. D-pen was conjugated to PGA-cystamide in the second step via thiol-disulfide exchange. To a solution of sodium PGA (20 mg, 0.67 μmol) in DMF-H₂O (4/1) was added N-hydroxy succinimide (NHS) (1.76 mg, 0.015 mmol), 1-Ethyl-3-[3-dimethylaminopropyl]carbodiimide Hydrochloride (EDC) (29.39 mg, 0.153 mmol), triethyl amine (1 mmol) and cystamine dihydrochloride (34.45 mg, 0.153 mmol). The reaction mixture was stirred for 2 h at room temperature. The solvent was removed by vacuum evaporation, the mixture reconstituted in 0.05 M Borate buffer pH 9.0 and PGA-cystamide was purified by Sephadex G-25 column. D-pen (34.32 mg, 0.23 mmol) was added to PGA-cystamide solution in 0.05 M Borate buffer pH 9.0 and the reaction mixture was left under constant stirring for 16 h at room temperature. PGA-D-pen conjugate was purified by Sephadex G-25 column.

To synthesize fluorescently labeled PGA-D-pen conjugate, 0.04 ml NHS-fluorescein in DMSO (3.2 mM) was added to 0.45 ml of PGA-D-pen conjugate in PBS buffer pH 7.4. The reaction mixture was stirred in dark for 1 h at room temperature. The fluorescently labeled conjugate was purified by Sephadex G-25 column. Moles of fluor per mole of PGA were determined spectrophotometrically (ε=68000 M⁻¹ cm⁻¹, λₘₐₓ=494 nm).

PGA Determination: The PGA concentration was analyzed using the BCA protein assay. Briefly, 50 μL of standard or sample was added to 150 μL of BCA Assay Reagent (Fisher Scientific), mixed and incubated for 2 h at 37° C. and the absorbance was read at 562 nm with the Synergy™ 2 Multi-Detection Microplate Reader (Biotek, Winooski, Vt.).

Measurement of Extent of Conjugation: 25 mM TCEP (0.18 ml) was added to PGA-D-pen conjugate (0.02 ml) and stirred for 1 h at room temperature. 20 μl of the reaction mixture was injected into HPLC to analyze the D-pen released from the conjugate. Total D-pen was expressed as combination of the yield of D-pen and D-pen disulfide.

HPLC Determination of D-pen: D-pen and D-pen disulfide released upon reduction of the PGA-D-pen conjugate in presence of TCEP were analyzed with a modification of our previously reported HPLC method. The HPLC analysis was preformed on Finnigan™ Surveyor HPLC System (Thermo Electron Corp., San Jose, Calif.) with a Gemini C18 column (250×4.6 mm; 5 μM; 20 μl sample; Phenomenex, Torrance, Calif.). The mobile phase employed was 20 mM ammonium dihydrogen phosphate+3% acetonitrile adjusted to pH 2.5 using o-phosphoric acid, and pumped at a flow rate of 1 mL/min. D-pen and D-pen disulfide were detected by UV absorption at 214 nm with retention times of 5.08 and 4.52 min, respectively.

Cell Culture and Cytotoxicity: The P388 cells and HL-60 cells were plated at 1×10⁴ and 4×10⁴ cells respectively in 200 μl of medium per well in round bottom 96-well microwell plates. The MDA-MB-468 cells were plated at 1×10⁴ cells/well in 96-well flat bottom microwell plates and allowed to attach overnight. Equal volumes of PGA-D-pen conjugate, PGA, D-pen, PGA+D-pen or PBS were added and the plates were incubated for 48 h (37° C., 5% CO₂). 20 μl of MTT reagent (5 mg/ml in PBS; Sigma) was added to each well and re-incubated for 3-4 h to allow formation of formazan crystals. The round bottom plates were centrifuged at 200 g for 5 min. Subsequently, the supernatant was aspirated and 200 μl of DMSO were added to each well and the plate was incubated at room temperature for 1 h to lyse the cells and solubilize formazan. The optical density of each well at 570 nm was measured on a Synergy™ 2 Multi-Detection Microplate Reader (Biotek, Winooski, Vt.). Percent viability in treated wells was calculated as the percentage of optical density in control wells.

Intracellular ROS Measurement: ROS generation was assessed using 5-(and-6)-carboxy-2′,7′-dichlorodihydrofluorescein diacetate (carboxy-H2DCFDA) (Molecular Probes). Carboxy-H2DCFDA is a cell permeant probe and has improved intracellular retention due to additional negative charges at cytosolic pH. It is converted to highly fluorescent form upon deacetylation by cellular esterases. Stock solution of carboxy-H2DCFDA (2 mM) was prepared in DMSO. Further dilutions were prepared in PBS. For ROS measurement, HL-60 cells were incubated for 30 min in PBS containing 25 μM carboxy-H2DCFDA. Subsequently, the cells were washed with PBS and resuspended in RPMI-1640 without phenol red and serum. 3×10⁴ cells were plated in 96-well flat bottom plates and treated with different concentrations of D-pen and PGA-D-pen conjugate. Fluorescence was determined at various time points post-treatment using Synergy™ 2 Multi-Detection Microplate Reader (Biotek, Winooski, Vt.) at excitation wavelength of 485±20 nm and an emission wavelength of 530±30 nm. RPMI-1640 and cells not incubated with the probe were used as negative controls while 100 μM H₂O₂ was used as positive control.

Confocal Microscopy: The uptake of the fluorescently labeled PGA-D-pen conjugate was determined qualitatively using confocal microscopy. HL-60 cells (5×10⁵) cultured in RPMI-1640 without phenol red and supplemented with 10% fetal calf serum, 100 units/ml penicillin and 100 μg/ml streptomycin, were plated in a 24 well plate and treated with fluorescently labeled PGA-D-pen conjugate. Cells were washed with PBS and resuspended in RPMI 1640 without phenol red and immediately taken for confocal microscopy. Cells were transferred onto a slide for visualizing using Zeiss 510 Meta Laser Scanning Confocal Microscope (63×1.4 NA oil Plan-Apochromat objective; excitation=488 nm and emission=515 nm; Carl Zeiss, Thornwood, N.Y.). Differential Interference Contrast (DIC) images, fluorescence images and the overlapped images taken from the microscope were visualized using the Zeiss AIM Viewer (Carl Zeiss, Thornwood, N.Y.).

Statistical Analysis. Statistical analysis was performed with GraphPad Prism® 4 Software (GraphPad software Inc. San Diego, Calif.). Results were depicted as mean±SEM. Cytotoxicity results were analyzed by two-way ANOVA followed by Bonferroni's multiple comparisons test. ROS generation was analyzed by one-way ANOVA followed by Dunnet's post test to compare the different dose levels to control.

Results

Synthesis and Characterization of PGA-D-pen Conjugate: PGA-D-pen conjugate was purified by Sephadex G-25 and analyzed for extent of conjugation by reduction of disulfide bonds and measuring the released D-pen by HPLC. The conjugate contained 33.12±2.51 mg D-pen/g PGA. ¹H NMR (Inova 500 spectrometer; 500 MHz in D₂O) of the conjugate showed resonance of D-pen at 3.67 ppm (C_{α}—H), 1.54 ppm (C_{β} methyl-H) and 1.46 ppm (C_{β} methyl-H) and resonance of PGA at 4.30 ppm (C_{α}—H), 2.25 ppm (C_{γ}—H) and 2.05 ppm (C_{β}—H).

Cytotoxicity of the PGA-D-pen Conjugate: The in-vitro cytotoxicity of the conjugate was investigated in leukemia (HL-60 and P388) and breast cancer cells (MDA-MB-468). The results were analyzed in terms of percentage of viable cells after 48 h of incubation as compared to control cells. Free D-pen or PGA, or D-pen+PGA did not cause significant reduction in cell viability over the duration of study. The PGA-D-pen conjugate treatment resulted in a dose-dependent reduction in viability of the cells (FIG. 2). The IC₅₀ values for HL-60 (4×10⁴ cells), P388 (1×10⁴ cells) and MDA-MB-468 (1×10⁴ cells) were 78.58±1.75 μM, 104±3.13 μM, 156.7±1.77 μM respectively.

FIG. 21 shows in vitro cytotoxicity of PGA-Dpen conjugate at 48 hr in a) HL-60 cells; b) P388 cells and c) MDA-MB-468 cells. The log of equivalent D-pen concentration was plotted on the X-axis.

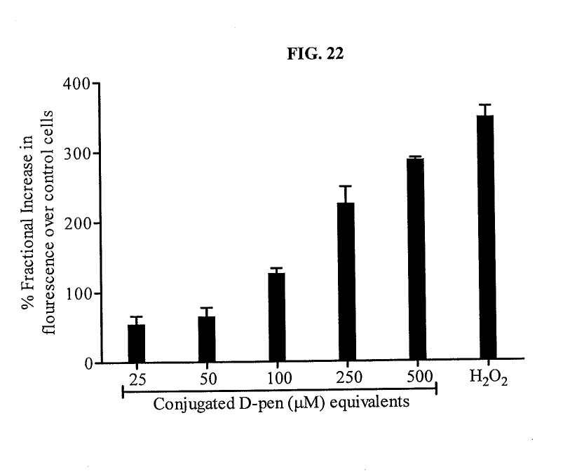

Intracellular ROS Generation: The generation of ROS upon release of D-pen from the conjugate was investigated using carboxy-H2DCFDA, a non-fluorescent probe which gets converted to highly fluorescent derivative following deacetylation by intracellular esterases. This dye was chosen due to its longer intracellular retention compared to H2DCFDA which was employed in our earlier studies. The conjugate is expected to gradually release D-pen following its uptake and this requires monitoring of cells for ROS generation for longer time. The time and dye concentration required for the study was optimized using H₂O₂, which was also used as a positive control. The ROS levels were significantly higher compared to the control at all concentrations tested (FIG. 22). The ROS levels at the highest concentration of the conjugate i.e. 500 μM (in terms of conjugated D-pen) were not significantly different from the levels produced by 100 μM H₂O₂ which suggests the strong potential of the synthesized conjugate to generate intracellular ROS upon release of D-pen.

Confocal Microscopy: The PGA-D-pen conjugate was fluorescently labeled using NHS-fluorescein to investigate the intracellular uptake by confocal microscopy. Live cells were visualized under the microscope after exposure to the conjugate for predetermined time. The conjugate exhibited time and dose dependent uptake in HL-60 cells. The control cells were visualized to confirm that the cells were healthy and to rule out any background fluorescence. Intracellular uptake of PGA-D-pen conjugate was determined using confocal microscopy. Fluorescence images, Differential Interference Contrast (DIC) images (top right), and the overlapped images (bottom) of cells were visualized by live microscopy without treatment or after treatment at 4 h and 24 h using PGA-D-pen conjugate (40 μM D-pen) fluorescently labeled using NHS-fluorescein.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,664,911
U.S. Pat. No. 4,680,338
U.S. Pat. No. 4,792,447
U.S. Pat. No. 4,870,287
U.S. Pat. No. 5,045,451
U.S. Pat. No. 5,141,648
U.S. Pat. No. 5,399,363
U.S. Pat. No. 5,401,511
U.S. Pat. No. 5,466,468
U.S. Pat. No. 5,543,158
U.S. Pat. No. 5,563,250
U.S. Pat. No. 5,578,706
U.S. Pat. No. 5,580,579
U.S. Pat. No. 5,603,872
U.S. Pat. No. 5,629,001
U.S. Pat. No. 5,641,515
U.S. Pat. No. 5,641,515
U.S. Pat. No. 5,686,072
U.S. Pat. No. 5,725,871
U.S. Pat. No. 5,756,353
U.S. Pat. No. 5,760,395
U.S. Pat. No. 5,767,072
U.S. Pat. No. 5,780,045
U.S. Pat. No. 5,792,451
U.S. Pat. No. 5,804,212
U.S. Pat. No. 5,889,155
U.S. Pat. No. 6,537,514
U.S. Pat. No. 6,613,308
U.S. Application 2005/0080132
U.S. Application 2007/0072800
U.S. Application 2003/0147844
U.S. Application 2003/0157052
Azarmi et al., J. Pharm. Pharmaceut. Sci., 9:124-132, 2006.
Behrend et al., Biochem. Soc. Trans., 31:1441-1444; 2003.
Bowman and Ofner, Pharm. Res., 17:1309-1315, 2000.
Brem et al., Neuro-Oncol., 7:246-253, 2005.
Brem, Cancer Control, 6:436-458, 1999.
Brewer, Drug Dis. Today, 10:1103-1109, 2005.
Cavallaro et al., J. Control Release, 115:322-334, 2006.
Chan and Wong, Ann. Clin. Biochem., 30:545-549, 1993.
Chen et al., Apoptosis, 10:1131-1142, 2005.
Chung et al., Biotech. Lett., 25:1993-1997, 2003.
Chvapil et al., Connective Tissue Res., 46:242-250, 2005.
Culver et al., Science, 256(5063):1550-1552, 1992.
Daniel et al., Breast Cancer Res., 7:R897—R908, 2005.
Diez et al., Oncology, 46:230-234, 1989.
Ding et al., Cancer Res., 65:3389-3395, 2005.
Goodman & Gilman's The Pharmacological Basis of Therapeutics
Guo-fu, J. Cell. Biochem., 69:326-335, 1998.
Gupte and Mumper, Free Rad. Biol. Med., 43:1271-1278, 2007b.
Gupte and Mumper, J. Inorg. Biochem., 101:594-602, 2007a.
Held and Biaglow, Rad. Res., 139:15-23, 1994.
Held et al., Rad. Res., 145:542-553, 1996.
Hileman et al., Cancer Chemother. Pharmacol., 53:209-219, 2003.
Hwang et al., Crit. Rev. Ther. Drug Carrier Syst., 15(3):243-284, 1998.
Jones et al., Free Rad. Biol. Med., 28:625-635, 2000.
Joyce, Bailliere's Clin. Rheum., 4:553-574, 1990.
Joyce, Pharmac. Ther., 42:405-427, 1989.
Kaul and Amiji, Pharm. Res., 19:1062-1068, 2002.
Kommareddy and Amiji, Bioconj. Chem., 16:1423-1432, 2005.
Kommareddy and Amiji, J. Pharm. Sci., 96:397-407, 2007.
Leonard et al., Free Rad. Med. Biol., 37:1921-1942, 2005.
Lodemann, Biochem. Biophys. Res. Commun., 102:775-783, 1981.
Lowndes and Harris, Oncol. Res., 14:529-539; 2004.
Lu and Combs, J. Nutr., 122:262-355, 1992.
Maeda et al., Cell Death Differ., 11:737-746, 2004.
Mamou et al., Anticancer Res., 26:1753-1758, 2006.
Matasubara and Hirohata, Arth. Rheum., 31:964-972, 1988.
Matasubara et al., J. Clin. Invest., 83:158-167, 1989.
Mathiowitz et al., Nature, 386(6623):410-414, 1997.
Munday, Free Rad. Med. Biol., 7:659-673, 1989.
Nakanishi et al., Nephron Exp. Nephrol., 100:11-20, 2005.
Nath and Salahudden, Am. J. Physiol., 264:F306-F314, 1993.
Netter et al., Clinical Pharmacokinetics, 13:317-333, 1987.
Ofner et al., Int. J. Pharm., 308:90-99, 2006.
Okroj et al., Cell. Mol. Biol. Lett., 11:384-395, 2006.
Pan et al., Can. Res., 62:4854-4859, 2002.
Pelicano et al., Drug Resistance Updates, 7:97-110, 2004.
Pelicano et al., J. Biol. Chem., 278:37832-37839, 2003.
Pervaiz and Clement, Curr. Pharm. Des., 10:1969-1977, 2004.
Physicians Desk Reference
Pica et al., J. Pharm. Sci., 95:1896-1908, 2006.
Raju et al., J. Natl. Cancer Ins., 69:1183-1188, 1982.
Remington: The Science and Practice of Pharmacy, 21^{st} edition, by University of the Sciences in Philadelphia
Remington's Pharmaceutical Sciences” 15^{th} Ed., 1035-1038 and 1570-1580, 1990.
Remington's Pharmaceutical Sciences, 18^{th} Ed. Mack Printing Company, 1289-1329, 1990.
Renschler, Eur. J. Cancer, 40:1934-1940, 2004.
Saito et al., Adv. Drug Delivery Rev., 55:199-215, 2003.
Samoszuk and Nguyen, Anticancer Res., 16:1219-1223, 1996.
Santoliquido et al., Sur. Gyn. Obs., 142:65-69, 1976.
Scanni et al., Tumori, 63:175-180, 1977.
Schafer and Buettner, Free Rad. Med. Biol., 30:1191-1212, 2001.
Schilsky, Sem. Liver Dis., 16:83-95, 1996.
Schwartz et al., Surgery, 76:325-329, 1974.
Starkebaum and Root, J. Immun., 134:3371-3378, 1985.
Takenaga et al., J. Control Release, 52(1-2):81-87 1998
The Merck Index, Eleventh Edition
Vande Stat et al., Scand. J. Rheum., 28:13-20, 1979.
Winterbourn and Metodiewa, Free Rad. Med. Biol., 27:322-328, 1999.
Young et al., J. Cont. Rel., 109:256-274, 2005.
Yucel et al., Biol. Trace Elem. Res., 40:31-37, 1994.
Zhou et al., Blood, 101:4098-4104, 2003.
Zuo et al., Biol. Trace Elem. Res., 114:41-54, 2006.

What is claimed is:

1. A composition comprising at least one D-penicillamine covalently bonded to a biocompatible polymer via a disulfide bond, wherein the disulfide bond comprises the sulfur group of D-penicillamine.

2. The composition of claim 1, wherein the polymer is gelatin.

3. The composition of claim 1, wherein the polymer is chitosan.

4. The composition of claim 1, wherein the polymer is polyglutamic acid

5. The composition of claim 1, wherein the polymer is selected from the group consisting of poly-L-lysine, poly-L-Arginine, albumin, N-(2-hydroxypropyl) methacrylamide (HPMA), polyaspartamide, a dendrimer comprising a polyamido amine and polylysine core, hyaluronic acid, polylactic-co-glycolic acid, heparin, polyacrylic acid, crosslinked polyacrylic acid, carboxymethylcellulose, alginate, alginic acid, propylene glycol alginate, sodium alginate, a polylactide, poly-glutamic acid, and polyerucic-co-sebacic acid.

6. The composition of claim 1, wherein said disulfide bond comprises a sulfur group present in the polymer, and wherein the D-penicillamine is covalently bonded directly to the polymer via the disulfide bond.

7. The composition of claim 1, wherein said disulfide bond comprises a sulfur group in a linker or a coupling agent, wherein the linker or coupling agent is covalently bonded to the polymer.

8. The composition of claim 7, wherein said linker is selected from the group consisting of SPDP, LC-SPDP, and Sulfo-LC-SPDP.

9. The composition of claim 1, wherein said polymer has at least about 20% of available functionalities occupied by D-penicillamine or a linker, wherein the linker is coupled to D-penicillamine.

10. The composition of claim 9, wherein said polymer has at least about 50% of available functionalities occupied by D-penicillamine or a linker.

11. The composition of claim 1, wherein the composition comprises gelatin-D-penicillamine.

12. The composition of claim 1, wherein the composition comprises chitosan-D-penicillamine.

13. The composition of claim 1, wherein the composition comprises polyglutamic acid-D-penicillamine.

14. The composition of claim 1, wherein the polymer is conjugated to an antibody.

15. The composition of claim 14, wherein the antibody selectively binds a protein whose expression is upregulated in a cancer.

16. The composition of claim 15, wherein the antibody is selected from the group consisting of CD123, Rituximab, Trastuzumab, Gemtuzamab, Alemtuzumab, Ibritumomab, Tositumomab, and Bevacizumab.

17. The composition of claim 1, wherein the polymer is conjugated to a polyethylene glycol having a molecular weight between about 2000 g/mol and about 20,000 g/mol.

18. The composition of claim 1, wherein the polymer is conjugated to an imaging agent.

19. The composition of claim 18, wherein the imaging agent is a fluorophore or a radioisotope.

20. The composition of claim 18, wherein the imaging agent is a photon emission computed tomography (PET) imaging agent or a single photon emission computed tomography (SPECT) imaging agent.

21. The composition of claim 1, wherein said composition is comprised in a pharmaceutically acceptable excipient.

22. The composition of claim 21, wherein said pharmaceutically acceptable excipient comprises a lipid, liposomes, or nanoparticles.

23. The composition of claim 21, wherein said pharmaceutically acceptable excipient is formulated for parenteral administration.

24. The composition of claim 23, wherein said pharmaceutically acceptable excipient is formulated for intravenous administration.

25. The composition of claim 23, wherein said pharmaceutically acceptable excipient is formulated for intratumoral injection.

26. A method of treating a cancer comprising administering a composition of claim 1 to a subject.

27. The method of claim 26, wherein said subject is a mammal.

28. The method of claim 27, wherein said mammal is a human patient.

29. The method of claim 26, wherein said composition is administered parenterally, intravenously, or intratumorally.

30. The method of claim 26, wherein said composition is administered at a dose of from about 1 microgram/kg body weight to about 1000 milligram/kg body weight.

31. The method of claim 26, wherein said method further comprises administering a second cancer therapy to the subject.

32. The method of claim 26, wherein the second cancer therapy is a chemotherapeutic, a surgery, a radiation therapy, an immunotherapy, or a gene therapy.

33. The method of claim 32, wherein said second cancer therapy is a chemotherapeutic selected from the list consisting of paclitaxel, docetaxel, doxorubicin, a platinum-containing chemotherapeutic, idarubicin, and 5-FU.

34. The method of claim 26, wherein said cancer is selected from the group consisting of leukemia, cancer of the lymph node or lymph system, bone cancer, cancer of the mouth or esophagus, stomach cancer, colon cancer, breast cancer, ovarian cancer, a gastric cancer, brain cancer, renal cancer, liver cancer, prostate cancer, melanoma, and lung cancer.
